(19) United States
(12) Patent Application Publication
(10) Pub. No.: US 2024/0342127 A1
(43) Pub. Date: Oct. 17, 2024

(54) METHODS OF TREATING COPPER METABOLISM-ASSOCIATED DISEASES OR DISORDERS

(71) Applicant: Alexion Pharmaceuticals, Inc., Boston, MA (US)

(72) Inventors: Eugene Scott Swenson, Madison, CT (US); Wei-Jian Pan, Pullman, WA (US)

(73) Assignee: Alexion Pharmaceuticals, Inc., Boston, MA (US)

(21) Appl. No.: 18/685,127

(22) PCT Filed: Sep. 6, 2022
(86) PCT No.: PCT/US2022/042664

§ 371 Date: Feb. 20, 2024

Related U.S. Application Data
(60) Provisional application No. 63/241,441, filed on Sep. 7, 2021.

(51) Int. Cl. A61K 31/28; G01N 33/68; G01N 33/84

(52) U.S. Cl. CPC A61K 31/28; G01N 33/6893; G01N 33/84; G01N 2333/90287

(57) ABSTRACT

Disclosed are methods of treating a copper metabolism-associated disease or disorder, such as Wilson disease (WD) in a subject, particularly in a subject who is from about 3 years old to less than about 18 years old. Also disclosed are compositions comprising bis-choline tetrathiomolybdate for use in the treatment of a copper metabolism-associated disease or disorder, such as Wilson disease (WD) in a subject, particularly in a subject who is from about 3 years old to less than about 18 years old. Also disclosed are uses of a composition comprising bis-choline tetrathiomolybdate for the manufacture of a medicament for treating a copper metabolism-associated disease or disorder, such as Wilson disease (WD) in a subject, particularly in a subject who is from about 3 years old to less than about 18 years old.

### CROSS REFERENCE TO RELATED APPLICATIONS

This application claims the benefit of U.S. Provisional Application No. 63/241,441, filed on Sep. 7, 2021, which is incorporated by reference herein in its entirety

### FIELD OF THE DISCLOSURE

This disclosure relates to methods of treating a copper metabolism-associated disease or disorder, such as Wilson disease (WD) in a subject, particularly in a subject who is from about 3 years old to less than about 18 years old. This disclosure also relates to compositions comprising bis-choline tetrathiomolybdate for use in the treatment of a copper metabolism-associated disease or disorder, such as Wilson disease (WD) in a subject, particularly in a subject who is from about 3 years old to less than about 18 years old. This disclosure further relates to uses of a composition comprising bis-choline tetrathiomolybdate for the manufacture of a medicament for treating a copper metabolism-associated disease or disorder, such as Wilson disease (WD) in a subject, particularly in a subject who is from about 3 years old to less than about 18 years old.

### BACKGROUND

Wilson disease (WD) is an autosomal recessive disorder of impaired copper transport. Mutations in the ATP7B gene result in deficient production of the copper-transporter ATPase2, leading to impaired incorporation of copper into ceruloplasmin (Cp), impaired biliary excretion of copper, increased exchangeable copper, and copper accumulation in liver, brain, and other tissues, with resulting organ damage and dysfunction. Ceruloplasmin is a serum ferroxidase, and in healthy humans, it contains greater than 95% of the copper found in plasma.

The prevalence of genetic markers associated with WD is approximately one per 30,000 population worldwide. Among people with an identified mutation, disease manifestation will be present in approximately 50%. A recent nationwide, population-based, epidemiological study based in France found the diagnosed prevalence of WD to be 1.5 per 100,000 population.

Typical clinical presentation of WD is in adolescence to early adulthood. Genetic screening and genotype-phenotype correlation is complicated by a multitude (>500) of associated ATP7B mutations; most individuals with WD are compound heterozygotes. Initial signs and symptoms of WD are predominantly hepatic (˜40%), neurologic (˜40%), or psychiatric (˜20%), but patients often develop combined hepatic and neuropsychiatric disease. Untreated or inadequately treated patients have progressive morbidity, and mortality is usually secondary to hepatic cirrhosis. Liver transplantation is the only effective therapy for WD-associated acute liver failure; other causes of death associated with WD include hepatic malignancy and neurologic deterioration with severe inanition.

The liver represents one of the main copper storage organs in humans. In healthy people, intracellular copper homeostasis is tightly regulated. Copper is transported into cells by copper transporter 1 (CTR1), and then transferred to copper chaperones such as the copper chaperones for antioxidant 1, cytochrome c oxidase, and superoxide dismutase. Copper accompanying the chaperone is delivered to a specific copper-requiring enzyme. If excess amounts of copper appear, then the excess copper is bound to metallothionein (MT) as monovalent copper (Cu+) via copper thiolate bridges by abundant cysteine residues in MT, thus leading to a detoxification of copper through a reduction of its redox potential.

In patients with WD, copper is not removed from the tissue compartments due to the deficient activity of ATPase2 due to its absence or reduced function. This results in an accumulation of copper, mainly in the liver where the protein is highly expressed in hepatocytes and then in the brain, but also in other organs. Within the capacity of MT biosynthesis, no apparent toxicity of copper exists because MT tightly binds copper.

However, beyond the copper buffering capacity of MT, free copper ions appear and this excessive amount of free intracellular copper triggers pro-oxidant properties, leading to an increased risk of tissue/organ damages with clinical manifestations as a result. Historically, it has been assumed that the hepatic toxicity of copper in WD is mediated by copper that is not bound to ceruloplasmin or MT. Increased non-ceruloplasmin-bound copper (NCC) from the liver then enters the circulation in a form that is mostly bound to albumin, and is available for uptake into other organs where it may cause damage. Therefore, plasma NCC (NCC) concentration may serve as an important biomarker for tissue copper overload. However, achieving a normalized plasma NCC concentration does not necessarily reflect normalized tissue copper levels, particularly in organs with relatively slow copper exchange, such as the brain.

An optimal treatment goal of an effective therapy for WD should be to remove excessive copper from the tissues or safely sequester it. The current treatments for WD are general chelator therapies D-penicillamine (Cuprimine, Depen) and trientine (Syprine), which non-specifically chelate copper and promote urinary copper excretion. In addition, zinc, which blocks dietary uptake of copper, is used mainly for maintenance treatment. Zinc impairs the absorption of copper by the induction of MT in the enterocytes of the gastrointestinal (GI) tract. As tissue copper concentrations are not readily sampled, the adequacy of therapeutic copper control is currently monitored through periodic assessment of the 24-hour urinary copper excretion. The daily urinary copper excretion rate and plasma NCC concentration are both highly variable and neither is ideal for monitoring therapeutic copper control.

Currently available drugs have high rates of treatment discontinuation due to adverse events (AEs) and treatment failure. They also need to be dosed 2 to 5 times per day and must be taken in the fasted state. Their AE profiles and complicated dosing regimens lead to poor treatment compliance and high rates of treatment failure, a major concern in a disease that requires life-long treatment such as WD.

Bis-choline tetrathiomolybdate (“BC-TTM”) (also known as ALXN1840, tiomolibdate choline, and tiomolibdic acid; formerly known as WTX101) is an investigational, oral, first-in-class copper-protein-binding molecule being developed for the treatment of WD. BC-TTM has the following structure:

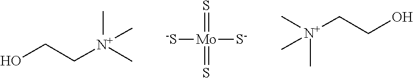

Prior work suggested that BC-TTM improves control of Cu due to rapid and irreversible formation of Cu-tetrathiomolybdate-albumin tripartite complexes (TPCs) leading to rapid sequestration of excess Cu without mobilization of free Cu that could cause tissue toxicity including neurological deterioration. It is hoped that improved long-term compliance with BC-TTM treatment through improved tolerability and the convenience of a simplified once daily (QD) dosing regimen compared with current therapeutic options could be achieved.

WD is most commonly diagnosed in teenagers and young adults and rarely presents symptomatically before 5 years of age. In a European cohort of 1357 patients, the average age at diagnosis of WD was 19.8 years (±10 years), and half of all patients were diagnosed before the age of 18. The age of presentation of WD is generally over 5 years. Based on the literature and feedback from leading EU and US reference clinical institutions managing pediatric patients with WD, there are exceptionally few diagnosed cases of WD in children <6 years of age. These exceptionally early newly diagnosed patients may include siblings or patients who may present symptoms as early as 3 years.

Standard treatments approved and used in adults with WD (penicillamine, trientine, zinc) are also approved for use in children and adolescents. However, significant unmet needs still exist with respect to efficacy, safety, tolerability, simplicity and frequency of dosing regimens, and especially with an appropriate compliance to therapy in young participants.

### SUMMARY OF THE DISCLOSURE

The disclosure generally provides methods useful for treating a copper metabolism-associated disease or disorder, such as Wilson disease, in a subject who is from about 3 years old to less than about 18 years old, such as, for example, a subject who is from about 3 years old to less than about 12 years old. Such methods include: administering to the subject a therapeutically effective amount of bis-choline tetrathiomolybdate.

The disclosure also provides a therapeutically effective amount of bis-choline tetrathiomolybdate for use in treating a copper metabolism-associated disease or disorder in a subject who is from about 3 years old to less than about 18 years old. For example, in certain embodiments the subject is from about 3 years old to less than about 12 years old.

In an aspect, this disclosure provides methods for treating a copper metabolism-associated disease or disorder in a subject, wherein the subject is from about 3 years old to less than about 18 years old, the method comprising administering to the subject a therapeutically effective amount of bis-choline tetrathiomolybdate.

In some embodiments of the methods disclosed herein, the copper metabolism-associated disease or disorder is Wilson Disease (WD).

In some embodiments of the methods disclosed herein, the subject is from about 3 years old to less than about 12 years old.

In some embodiments of the methods disclosed herein, the therapeutically effective amount of bis-choline tetrathiomolybdate is in the range of about 2.5 mg to about 15 mg per day. In certain embodiments, the therapeutically effective amount of bis-choline tetrathiomolybdate is about 2.5 mg per day. In certain embodiments, the therapeutically effective amount of bis-choline tetrathiomolybdate is about 5 mg per day. In certain embodiments, the therapeutically effective amount of bis-choline tetrathiomolybdate is in the range of about 5 mg to about 15 mg per day. In certain embodiments, the therapeutically effective amount of bis-choline tetrathiomolybdate of about 2.5 mg per day is maintained for at least 4 weeks. In certain embodiments, wherein, after at least 4 weeks, the therapeutically effective amount of bis-choline tetrathiomolybdate of about 2.5 mg per day is increased to a second therapeutically effective amount of bis-choline tetrathiomolybdate. In certain embodiments, the increase to the second therapeutically effective amount of bis-choline tetrathiomolybdate is in one or more increments of 2.5 mg per day, each increment at least 4 weeks apart.

In some embodiments of the methods disclosed herein, the subject is from about 12 years old to less than about 18 years old. In certain embodiments, the therapeutically effective amount of bis-choline tetrathiomolybdate is in the range of about 15 mg every other day to about 15 mg per day. In certain embodiments, the therapeutically effective amount of bis-choline tetrathiomolybdate is about 15 mg every other day. In certain embodiments, the therapeutically effective amount of bis-choline tetrathiomolybdate is about 15 mg per day. In certain embodiments, the therapeutically effective amount of bis-choline tetrathiomolybdate of about 15 mg every other day is maintained for at least 4 weeks, for at least 8 weeks, for at least 12 weeks, for at least 24 weeks, for at least 48 weeks, for at least 72 weeks, or for at least 96 weeks, or about 2 years, about 3 years, about 4 years, about 5 years, about 6 years, about 7 years, about 8 years, about 9 years, or about 10 years or longer. In certain embodiments, the therapeutically effective amount of bis-choline tetrathiomolybdate of about 15 mg per day is maintained for at least 4 weeks, for at least 8 weeks, for at least 12 weeks, for at least 24 weeks, for at least 48 weeks, for at least 72 weeks, or for at least 96 weeks, or about 2 years, about 3 years, about 4 years, about 5 years, about 6 years, about 7 years, about 8 years, about 9 years, or about 10 years or longer.

In some embodiments of the methods disclosed herein, the subject previously received no treatment for the copper metabolism-associated disease or disorder, such as for Wilson Disease (i.e., a treatment-naïve subject).

In some embodiments of the methods disclosed herein, the subject previously received a standard of care treatment for the copper metabolism-associated disease or disorder, such as for Wilson Disease. In certain embodiments, the subject previously received standard of care treatment for less than 28 days. In certain embodiments, the subject previously received standard of care treatment for at least 28 days, for at least 5 weeks, for at least 6 weeks, for at least 12 weeks, for at least 24 weeks, or for at least 48 weeks.

In some embodiments of the methods disclosed herein, the subject previously received no treatment or the subject previously received a standard of care treatment for less than 28 days for the copper metabolism-associated disease or disorder, such as for Wilson Disease. In certain embodiments, the standard of care treatment comprises trientine, D-penicillamine, and/or zinc. In certain embodiments, the standard of care treatment comprises trientine and/or D-penicillamine. In certain embodiments, the subject received a last dose of the standard of care treatment at least 2 weeks prior to administering bis-choline tetrathiomolybdate.

In some embodiments of the methods disclosed herein, the methods further comprise determining a concentration of one or more of total copper, ceruloplasmin, ceruloplasmin-bound copper (CpC), non-ceruloplasmin-bound copper (such as calculated, cNCC, or directly measured, dNCC), and labile-bound copper (LBC) in the subject's plasma.

In some embodiments of the methods disclosed herein, the methods further comprise determining a concentration of NCC_{corrected}; or determining a daily mean area under the effect-time curve (AUEC) of directly measured non-ceruloplasmin-bound copper (dNCC) (e.g., from baseline to 48 weeks). In certain embodiments, the methods further comprise adjusting the therapeutically effective amount of bis-choline tetrathiomolybdate if the subject's NCC_{corrected} is outside a reference range for NCC_{corrected}. In certain embodiments, the reference range for NCC_{corrected} is 0.8 to 2.3 μM.

In some embodiments of the methods disclosed herein, the methods further comprise determining a concentration of total molybdenum and/or plasma ultrafiltrate (PUF) molybdenum in the subject's plasma. In certain embodiments, the determining is performed at baseline, at or after 6 weeks of administration, at or after 24 weeks of administration, and/or at or after 48 weeks of administration. In certain embodiments, the determining is performed at baseline, and to 6 weeks of administration, or to 24 weeks of administration, or to 48 weeks of administration, or to at least 48 weeks or more of administration.

In some embodiments of the methods disclosed herein, the methods further comprise evaluating the patients for improvements in disability and neurologic symptoms as measured according to Unified Wilson Disease Rating Scale (UWDRS), part II, and/or part Ill.

In some embodiments of the methods disclosed herein, the methods further comprise evaluating the patients for improvements in disability status, psychiatric symptoms, clinical symptoms, treatment satisfaction, or a combination thereof.

In another aspect, the disclosure provides a composition comprising bis-choline tetrathiomolybdate for use in the treatment of a copper metabolism-associated disease or disorder in a subject, wherein the subject is from about 3 years old to less than about 18 years old.

In some embodiments of the compositions disclosed herein, the copper metabolism-associated disease or disorder is Wilson Disease (WD). In certain embodiments, the subject is from about 3 years old to less than about 12 years old. In certain embodiments, about 2.5 mg to about 15 mg per day of bis-choline tetrathiomolybdate is administered to the subject.

In certain embodiments, about 2.5 mg per day of bis-choline tetrathiomolybdate is administered to the subject. In certain embodiments, about 5 mg per day of bis-choline tetrathiomolybdate is administered to the subject. In certain embodiments, about 5 mg to about 15 mg per day of bis-choline tetrathiomolybdate is administered to the subject. In certain embodiments, about 2.5 mg per day of bis-choline tetrathiomolybdate is administered to the subject for at least 4 weeks. In certain embodiments, the amount of bis-choline tetrathiomolybdate that is administered to the subject is increased after at least 4 weeks. In certain embodiments, the amount of bis-choline tetrathiomolybdate is increased by one or more increments of 2.5 mg per day. In certain embodiments, the subject is from about 12 years old to less than about 18 years old. In certain embodiments, about 15 mg every other day to about 15 mg per day of bis-choline tetrathiomolybdate is administered to the subject. In certain embodiments, about 15 mg of bis-choline tetrathiomolybdate is administered to the subject every other day. In certain embodiments, about 15 mg per day of bis-choline tetrathiomolybdate is administered to the subject. In certain embodiments, about 15 mg of bis-choline tetrathiomolybdate is administered to the subject every other day for at least 4 weeks, for at least 8 weeks, for at least 12 weeks, for at least 24 weeks, for at least 48 weeks, for at least 72 weeks, or for at least 96 weeks, or about 2 years, about 3 years, about 4 years, about 5 years, about 6 years, about 7 years, about 8 years, about 9 years, or about 10 years or longer. In certain embodiments, about 15 mg per day of bis-choline tetrathiomolybdate is administered to the subject for at least 4 weeks, for at least 8 weeks, for at least 12 weeks, for at least 24 weeks, for at least 48 weeks, for at least 72 weeks, or for at least 96 weeks, or about 2 years, about 3 years, about 4 years, about 5 years, about 6 years, about 7 years, about 8 years, about 9 years, or about 10 years or longer.

In some embodiments of the compositions disclosed herein, the subject previously received no treatment for the copper metabolism-associated disease or disorder, such as for Wilson Disease, (i.e., a treatment-naïve subject).

In some embodiments of the compositions disclosed herein, the subject previously received a standard of care treatment for the copper metabolism-associated disease or disorder, such as for Wilson Disease. In certain embodiments, the subject previously received standard of care treatment for less than 28 days. In certain embodiments, the subject previously received standard of care treatment for at least 28 days, for at least 5 weeks, for at least 6 weeks, for at least 12 weeks, for at least 24 weeks, or for at least 48 weeks.

In some embodiments of the compositions disclosed herein, the subject previously received no treatment or the subject previously received a standard of care treatment for less than 28 days for the copper metabolism-associated disease or disorder, such as for Wilson Disease. In certain embodiments, the standard of care treatment comprises trientine, D-penicillamine, and/or zinc. In certain embodiments, the standard of care treatment comprises trientine and/or D-penicillamine. In certain embodiments, the subject received a last dose of the standard of care treatment at least 2 weeks prior to receiving bis-choline tetrathiomolybdate.

In some embodiments of the compositions disclosed herein, the concentration of one or more of total copper, ceruloplasmin, ceruloplasmin-bound copper (CpC), non-ceruloplasmin-bound copper (such as calculated, cNCC, or directly measured, dNCC), and labile-bound copper (LBC) in the subject's plasma is determined.

In some embodiments of the compositions disclosed herein, the concentration of NCC_{corrected} or the daily mean area under the effect-time curve (AUEC) of directly measured non-ceruloplasmin-bound copper (dNCC) (e.g., from baseline to 48 weeks) is determined. In certain embodiments, the amount of bis-choline tetrathiomolybdate is adjusted if the subject's NCC_{corrected} is outside a reference range for NCC_{corrected}. In certain embodiments, the reference range for NCC_{corrected} is 0.8 to 2.3 μM.

In some embodiments of the compositions disclosed herein, the concentration of total molybdenum and/or plasma ultrafiltrate (PUF) molybdenum in the subject's plasma is determined. In certain embodiments, the concentration of one or more of total copper, ceruloplasmin, ceruloplasmin-bound copper (CpC), non-ceruloplasmin-bound copper, and labile-bound copper (LBC) in the subject's plasma is determined at baseline, at or after 6 weeks of administration of bis-choline tetrathiomolybdate, at or after 24 weeks of administration of bis-choline tetrathiomolybdate, and/or at or after 48 weeks of administration of bis-choline tetrathiomolybdate. In certain embodiments, the concentration of one or more of total copper, ceruloplasmin, ceruloplasmin-bound copper (CpC), non-ceruloplasmin-bound copper, and labile-bound copper (LBC) in the subject's plasma is determined at baseline, and to 6 weeks of administration of bis-choline tetrathiomolybdate, or to 24 weeks of administration of bis-choline tetrathiomolybdate, or to 48 weeks of administration, or to at least 48 weeks or more of administration of bis-choline tetrathiomolybdate.

In some embodiments of the compositions disclosed herein, the subject is evaluated for improvements in disability and neurologic symptoms as measured according to Unified Wilson Disease Rating Scale (UWDRS), part II, and/or part III.

In some embodiments of the compositions disclosed herein, the subject is evaluated for improvements in disability status, psychiatric symptoms, clinical symptoms, treatment satisfaction, or a combination thereof.

In another aspect, this disclosure provides for the use of a composition comprising bis-choline tetrathiomolybdate for the manufacture of a medicament for treating a copper metabolism-associated disease or disorder in a subject, wherein the subject is from about 3 years old to less than about 18 years old.

In some embodiments of the use of the compositions disclosed herein, the copper metabolism-associated disease or disorder is Wilson Disease (WD). In certain embodiments, the subject is from about 3 years old to less than about 12 years old. In certain embodiments, about 2.5 mg to about 15 mg per day of bis-choline tetrathiomolybdate is administered to the subject. In certain embodiments, about 2.5 mg per day of bis-choline tetrathiomolybdate is administered to the subject. In certain embodiments, about 5 mg per day of bis-choline tetrathiomolybdate is administered to the subject. In certain embodiments, about 5 mg to about 15 mg per day of bis-choline tetrathiomolybdate is administered to the subject. In certain embodiments, about 2.5 mg per day of bis-choline tetrathiomolybdate is administered to the subject for at least 4 weeks. In certain embodiments, the amount of bis-choline tetrathiomolybdate that is administered to the subject is increased after at least 4 weeks. In certain embodiments, the amount of bis-choline tetrathiomolybdate is increased by one or more increments of 2.5 mg per day. In certain embodiments, the subject is from about 12 years old to less than about 18 years old. In certain embodiments, about 15 mg every other day to about 15 mg per day of bis-choline tetrathiomolybdate is administered to the subject. In certain embodiments, about 15 mg of bis-choline tetrathiomolybdate is administered to the subject every other day. In certain embodiments, about 15 mg per day of bis-choline tetrathiomolybdate is administered to the subject. In certain embodiments, about 15 mg of bis-choline tetrathiomolybdate is administered to the subject every other day for at least 4 weeks, for at least 8 weeks, for at least 12 weeks, for at least 24 weeks, for at least 48 weeks, for at least 72 weeks, or for at least 96 weeks, or about 2 years, about 3 years, about 4 years, about 5 years, about 6 years, about 7 years, about 8 years, about 9 years, or about 10 years or longer. In certain embodiments, about 15 mg per day of bis-choline tetrathiomolybdate is administered to the subject for at least 4 weeks, for at least 8 weeks, for at least 12 weeks, for at least 24 weeks, for at least 48 weeks, for at least 72 weeks, or for at least 96 weeks, or about 2 years, about 3 years, about 4 years, about 5 years, about 6 years, about 7 years, about 8 years, about 9 years, or about 10 years or longer.

In some embodiments of the use of the compositions disclosed herein, the subject previously received no treatment for the copper metabolism-associated disease or disorder, such as for Wilson Disease, (i.e., a treatment-naïve subject).

In some embodiments of the use of the compositions disclosed herein, the subject previously received a standard of care treatment for the copper metabolism-associated disease or disorder, such as for Wilson Disease. In certain embodiments, the subject previously received standard of care treatment for less than 28 days. In certain embodiments, the subject previously received standard of care treatment for at least 28 days, for at least 5 weeks, for at least 6 weeks, for at least 12 weeks, for at least 24 weeks, or for at least 48 weeks.

In some embodiments of the use of the compositions disclosed herein, the subject previously received no treatment or the subject previously received a standard of care treatment for less than 28 days for the copper metabolism-associated disease or disorder, such as for Wilson Disease. In certain embodiments, the standard of care treatment comprises trientine, D-penicillamine, and/or zinc. In certain embodiments, the standard of care treatment comprises trientine and/or D-penicillamine. In certain embodiments, the subject received a last dose of the standard of care treatment at least 2 weeks prior to receiving bis-choline tetrathiomolybdate.

In some embodiments of the use of the compositions disclosed herein, the concentration of one or more of total copper, ceruloplasmin, ceruloplasmin-bound copper (CpC), non-ceruloplasmin-bound copper (such as calculated, cNCC, or directly measured, dNCC), and labile-bound copper (LBC) in the subject's plasma is determined.

In some embodiments of the use of the compositions disclosed herein, the concentration of NCC_{corrected} or the daily mean area under the effect-time curve (AUEC) of directly measured non-ceruloplasmin-bound copper (dNCC) (e.g., from baseline to 48 weeks) is determined. In certain embodiments, the amount of bis-choline tetrathiomolybdate is adjusted if the subject's NCC_{corrected} is outside a reference range for NCC_{corrected}. In certain embodiments, the reference range for NCC_{corrected} is 0.8 to 2.3 μM.

In some embodiments of the use of the compositions disclosed herein, the concentration of total molybdenum and/or plasma ultrafiltrate (PUF) molybdenum in the subject's plasma is determined. In certain embodiments, the concentration of one or more of total copper, ceruloplasmin, ceruloplasmin-bound copper (CpC), non-ceruloplasmin-bound copper, and labile-bound copper (LBC) in the subject's plasma is determined at baseline, at or after 6 weeks of administration of bis-choline tetrathiomolybdate, at or after 24 weeks of administration of bis-choline tetrathiomolybdate, and/or at or after 48 weeks of administration of bis-choline tetrathiomolybdate. In certain embodiments, the concentration of one or more of total copper, ceruloplasmin, ceruloplasmin-bound copper (CpC), non-ceruloplasmin-bound copper, and labile-bound copper (LBC) in the subject's plasma is determined at baseline, and to 6 weeks of administration of bis-choline tetrathiomolybdate, or to 24 weeks of administration of bis-choline tetrathiomolybdate, or to 48 weeks of administration, or to at least 48 weeks or more of administration of bis-choline tetrathiomolybdate.

In some embodiments of the use of the compositions disclosed herein, the subject is evaluated for improvements in disability and neurologic symptoms as measured according to Unified Wilson Disease Rating Scale (UWDRS), part II, and/or part III.

In some embodiments of the use of the compositions disclosed herein, the subject is evaluated for improvements in disability status, psychiatric symptoms, clinical symptoms, treatment satisfaction, or a combination thereof.

These and other features and advantages of the claimed invention will be more fully understood from the following detailed description taken together with the accompanying claims. It is noted that the scope of the claims is defined by the recitations therein and not by the specific discussion of features and advantages set forth in the present description.

### BRIEF DESCRIPTION OF THE DRAWINGS

FIG. 1 provides a schematic view of the study design

FIG. 2: Projected Pharmacokinetic Exposure Range for Participants Aged 12 to <18 Years. Simulations are based on a 2-compartmental population-PK model for pediatric participants based on allometric scaling of adult population-PK model currently under development using Day 1 PK data from Study 201 in participants with WD and relevant PK data from Study 102 in healthy volunteers. Simulations results will be updated once final data are available from the Study 201. Abbreviations: hr=hour; Mo=molybdenum; PK=pharmacokinetic; WD=Wilson Disease; yrs=years.

FIG. 3: Projected Pharmacokinetic Exposure Range for Patients Aged 3 to <12 Years Simulations are based on a 2-compartmental population-PK model for pediatric participants based on allometric scaling of adult population-PK model currently under development using Day 1 PK data from Study −201 in patients with WD and relevant PK data from Study −102 in healthy volunteers. Simulations results will be updated once final data are available from Study −201. Abbreviations: hr=hour; PK=pharmacokinetic; yrs=years; QD=once daily; QOD=every other day.

FIG. 4: Projected Pharmacokinetic Exposure Range for Participants Aged 3 to <6 Years.

### DETAILED DESCRIPTION OF THE DISCLOSURE

Before the disclosed processes and materials are described, it is to be understood that the aspects described herein are not limited to specific embodiments, and as such can, of course, vary. It is also to be understood that the terminology used herein is for the purpose of describing particular aspects only and, unless specifically defined herein, is not intended to be limiting.

Before describing the present disclosure in detail, a number of terms will be defined. Unless otherwise required by context, singular terms shall include pluralities and plural terms shall include the singular. For example, the singular forms “a,” “an,” and “the” include plural referents unless the context clearly dictates otherwise. It should be understood that the terms “a” and “an” as used herein refer to “one or more” of the enumerated components unless otherwise indicated or dictated by its context. The use of the alternative (e.g., “or”) should be understood to mean either one, both, or any combination thereof of the alternatives unless otherwise indicated.

In the present disclosure, any concentration range, percentage range, ratio range, or integer range is to be understood to include the value of any integer within the recited range and, when appropriate, fractions thereof (such as one tenth and one hundredth of an integer), unless otherwise indicated.

As used herein, the terms “about” and “approximately,” when used to a modify numeric value or numeric range, indicate that reasonable deviations from the value or range, typically 5% or 10% above and 5% or 10% below the value or range, remain within the intended meaning of the recited value or range. In certain embodiments, when values are expressed as approximations, by use of the antecedent “about,” it will be understood that the particular value forms one possible embodiment and variation of the given value is possible (e.g., about 80 may include 80±10%). It will be further understood that the endpoints of each of the ranges are significant both in relation to the other endpoint, and independently of the other endpoint.

It is noted that terms like “preferably,” “commonly,” and “typically” are not utilized herein to limit the scope of the claimed subject matter or to imply that certain features are critical, essential, or even important to the structure or function of the claimed subject matter. Rather, these terms are merely intended to highlight alternative or additional features that can or cannot be utilized in a particular embodiment of the present disclosure.

As used herein, the terms “prevent,” “preventing,” and “prevention” in the context of the administration of a therapy to a subject refer to the inhibition of the onset or recurrence of a disease or disorder in a subject.

As used herein, the terms “manage,” “managing,” and “management,” in the context of the administration of a therapy to a subject, refer to the beneficial effects that a subject derives from a therapy, which does not result in a cure of a disease or disorder. In certain embodiments, a subject is administered one or more therapies to “manage” a disease or disorder so as to prevent the progression or worsening of symptoms associated with a disease or disorder.

For the purposes of describing and defining the present disclosure it is noted that the term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

Unless expressly specified otherwise, the term “comprising” is used in the context of the present disclosure to indicate that further members may optionally be present in addition to the members of the list introduced by “comprising”. It is, however, contemplated as a specific embodiment of the present disclosure that the term “comprising” encompasses the possibility of no further members being present, i.e., for the purpose of this embodiment “comprising” is to be understood as having the meaning of “consisting of”.

As utilized in accordance with the present disclosure, unless otherwise indicated, all technical and scientific terms shall be understood to have the same meaning as commonly understood by one of ordinary skill in the art.

In view of the present disclosure, the methods and compositions described herein can be configured by the person of ordinary skill in the art to meet the desired need. The present disclosure provides improvements in treating copper metabolism-associated diseases or disorders.

In certain embodiments of the methods or BC-TTM of the disclosure as described herein, the copper metabolism associated disease or disorder is Wilson disease.

In certain embodiments, the copper metabolism associated disease or disorder is copper toxicity (e.g., from high exposure to copper sulfate fungicides, ingesting drinking water high in copper, overuse of copper supplements, etc.). In certain embodiments, the copper metabolism associated disease or disorder is copper deficiency, Menkes disease, or aceruloplasminemia. In certain embodiments, the copper metabolism associated disease or disorder is at least one selected from academic underachievement, acne, attention-deficit/hyperactivity disorder, amyotrophic lateral sclerosis (ALS), atherosclerosis, autism, Alzheimer's disease, Candida overgrowth, chronic fatigue, cirrhosis, depression, elevated adrenaline activity, elevated cuproproteins, elevated norepinephrine activity, emotional meltdowns, fibromyalgia, frequent anger, geriatric-related impaired copper excretion, high anxiety, hair loss, hepatic disease, hyperactivity, hypothyroidism, intolerance to estrogen, intolerance to birth control pills, Kayser-Fleischer rings, learning disabilities, low dopamine activity, multiple sclerosis, neurological problems, oxidative stress, Parkinson's disease, poor concentration, poor focus, poor immune function, ringing in ears, allergies, sensitivity to food dyes, sensitivity to shellfish, skin metal intolerance, skin sensitivity, sleep problems, and white spots on fingernails.

As used herein, the terms “treatment” and “treating” refer to (i) ameliorating the referenced disease state, condition, or disorder (or a symptom thereof), such as, for example, ameliorating a disease, condition or disorder in an individual who is experiencing or displaying the pathology or symptomatology of the disease, condition or disorder (i.e., reversing or improving the pathology and/or symptomatology) such as decreasing the severity of disease or symptom thereof, or inhibiting the progression of disease; or (ii) eliciting the referenced biological effect.

As provided herein, bis-choline tetrathiomolybdate (also known as BC-TTM, ALXN1840, tiomolibdate choline, tiomolibdic acid, and WTX101) is administered in the methods of the disclosure.

BC-TTM is a first-in-class, Cu-protein binding agent in development for the treatment of WD and has been described in detail in International Publication No. WO 2019/110619, which is incorporated by reference herein in its entirety. BC-TTM monotherapy has been evaluated in 28 patients with WD, where it was shown that BC-TTM reduced mean serum non-ceruloplasmin-bound Cu (NCC) by 72% at Week 24 compared with baseline.

Treatment with BC-TTM was generally well-tolerated, with most reported adverse events (AEs) being mild (Grade 1) to moderate (Grade 2). The most frequently reported drug-related AEs were changes in hematological parameters, fatigue, sulphur eructations, and other gastrointestinal symptoms. Reversible liver function test elevations were observed in 39% of patients; these elevations were mild to moderate, asymptomatic, were associated with no notable increases in bilirubin, and normalized with dose reduction or treatment interruption. No paradoxical neurological worsening was observed upon treatment initiation with BC-TTM.

In certain embodiments, for participants aged 12 to <18 years, a BC-TTM dose of 15 mg/day is administered, and dose escalation is not permitted. In some embodiments, BC-TTM doses can range from 15 mg every other day to 15 mg/day, and doses of <15 mg every other day may be considered. In certain embodiments, BC-TTM can be administered as 12×1.25 mg doses for a 15 mg total dose. In some embodiments, BC-TTM can be administered as 1×15 mg dose. In some embodiments, a dose can be 1 mg, 1.25 mg, 2 mg, 2.5 mg, 3 mg, 4 mg, 5 mg, 6 mg, 7 mg, 7.5 mg, 8 mg, 9 mg, 10 mg, 11 mg, 12 mg, 13 mg, 14 mg, or 15 mg, administered daily or every other day. In certain embodiments, the maximal dose for participants aged 12 to <18 years is 15 mg/day. In certain embodiments, the BC-TTM dose can be modified, reduced, temporarily interrupted, or restriction of dose increases as needed following review of results from one or more laboratory tests. For example, a dose may be increased or decreased following one or more laboratory tests for ALT, triglycerides, cholesterol, hemoglobin, platelets, neutrophils, or bilirubin. Specific criteria for dose reduction, temporary interruption of dosing, or restriction of dose increases of BC-TTM are detailed in Table 3.

In certain embodiments, for participants aged 3 to <12 years, a BC-TTM dose of 2.5 mg/day is administered, and dose escalation is permitted, but not required. The dose may be increased in increments of 2.5 mg daily with the permission of the Alexion Medical Monitor, depending on the participant's clinical status, NCC_{coorrected} concentrations, and safety laboratory results. Dose increases can occur at least 4 weeks apart and may only occur if no other dose modification (reduction or interruption) criteria apply. In certain embodiments, BC-TTM can be administered as 12×1.25 mg doses for a 15 mg total dose. In some embodiments, BC-TTM can be administered as 1×15 mg dose. In some embodiments, a dose can be 1 mg, 1.25 mg, 2 mg, 2.5 mg, 3 mg, 4 mg, 5 mg, 6 mg, 7 mg, 7.5 mg, 8 mg, 9 mg, 10 mg, 11 mg, 12 mg, 13 mg, 14 mg, or 15 mg, administered daily or every other day. In certain embodiments, the maximal dose for participants aged 3 to <12 years is 15 mg/day. In certain embodiments, the BC-TTM dose can be modified, reduced, temporarily interrupted, or restriction of dose increases as needed following review of results from one or more laboratory tests. For example, a dose may be increased or decreased following one or more laboratory tests for ALT, triglycerides, cholesterol, hemoglobin, platelets, neutrophils, or bilirubin. Specific criteria for dose reduction, temporary interruption of dosing, or restriction of dose increases of BC-TTM are detailed in Table 4.

In certain embodiments, a dose of BC-TTM can be administered in an oral, parenteral, rectal or transdermal dosage form. For oral administration a dose may take the form of solutions, suspensions, tablets, pills, capsules, powders, and the like. For example, tablets may comprise a dose of 1 mg, 1.25 mg, 2 mg, 2.5 mg, 3 mg, 4 mg, 5 mg, 6 mg, 7 mg, 7.5 mg, 8 mg, 9 mg, 10 mg, 11 mg, 12 mg, 13 mg, 14 mg, or 15 mg. In certain embodiments, multiple doses of BC-TTM can be administered, for example, daily, every other day, bi-weekly, weekly, bi-monthly, monthly, or more or less often, as needed, for a time period sufficient to achieve the desired response. In certain embodiments, a patient can be administered BC-TTM for about 4 weeks, about 8 weeks, about 12 weeks, about 24 weeks, about 48 weeks, about 72 weeks, about 96 weeks, or about 2 years, about 3 years, about 4 years, about 5 years, about 6 years, about 7 years, about 8 years, about 9 years, about 10 years or longer. In certain embodiments, the maximal dose for all subjects is 15 mg/day. In some embodiments, a BC-TTM dose may be administered with a small amount of food (e.g., apple sauce or yogurt). In certain embodiments, doses may be in the form of enteric-coated tablets.

As used herein, the terms “individual,” “patient,” “participant,” or “subject” are used interchangeably, and refer to any animal, including mammals, and, in at least one embodiment, humans. In certain embodiments, the subject is a healthy subject. In certain embodiments, the subject suffers from WD. In certain embodiments of the methods or BC-TTM of the disclosure as described herein, the subject has cirrhosis. In certain other embodiments, the subject does not have cirrhosis.

The methods or BC-TTM of the disclosure are useful as a first line treatment. Thus, in certain embodiments of the methods or BC-TTM of the disclosure, the subject previously received no treatment for Wilson disease (i.e., a treatment-naïve subject).

The methods or BC-TTM of the disclosure are also useful as a second line treatment and/or a first line maintenance treatment of WD. Thus, in certain embodiments of the methods or BC-TTM of the disclosure, the subject has previously received a standard of care (SoC) treatment for WD. For example, in certain embodiments, the subject has previously received trientine (also known as triethylenetatramine; N′-[2-(2-aminoethylamino)ethyl]ethane-1,2-diamine). Trientine may be sold under name CUPRIOR® (GMP-Orphan United Kingdom Ltd), SYPRINE® (Aton Pharma, Inc.), or Cufence (Univar, Inc.). In certain other embodiments, the subject has previously received trientine and zinc. In certain embodiments, the subject has previously received D-penicillamine (also known as penicillamine; (2S)-2-amino-3-methyl-3-sulfanylbutanoic acid). D-penicillamine may be sold under name CUPRIMINE® (Valeant Pharmaceuticals) or DEPEN® (Meda Pharmaceuticals). In certain other embodiments, the subject has previously received D-penicillamine and zinc. In certain embodiments, the subject has previously received zinc. In certain embodiments, the subject has previously received trientine, D-penicillamine, and/or zinc. In certain other embodiments, the subject has previously received trientine and/or D-penicillamine.

In certain embodiments of the methods or BC-TTM of the disclosure, the subject has received standard of care treatment for WD for no more than 4 weeks.

In certain embodiments of the methods or BC-TTM of the disclosure, the subject has received standard of care treatment for WD for at least 4 weeks. In certain embodiments, the standard of care treatment was at least 6 weeks, or at least 12 weeks, or at least 24 weeks, or at least 36 weeks, or at least 48 weeks, or at least 52 weeks long. In certain embodiments, the standard of care treatment was at least 41 months. In certain embodiments, the standard of care treatment was about 41 months to about 228 months. In certain embodiments, the standard of care treatment was at least 116 months. In certain embodiments, the standard of care treatment was at least 155 months.

The standard of care treatment need not be continuous. For example, the subject may receive the treatment on-and-off totaling at least 4 weeks (e.g., at least 6, or at least 12, or at least 24, or at least 36, or at least 48, or at least 50 or at least 52 weeks or at least 103 weeks, or at least 41 months, or about 41 months to about 228 months, or at least 116 months, or at least 155) of treatment. In certain embodiments, however, the standard of care treatment is continuous.

In certain embodiments of the methods or BC-TTM of the disclosure, the subject previously received no treatment or the subject previously received a standard of care treatment for no more than 4 weeks for the copper metabolism-associated disease or disorder, such as for Wilson disease.

In the methods or BC-TTM of the disclosure as described herein, the subject completed the standard of care treatment at least 2 weeks prior to administering bis-choline tetrathiomolybdate. In certain embodiments, the subject completed the standard of care treatment at least 3 weeks, at least 4 weeks, or at least 6 weeks prior to administering bis-choline tetrathiomolybdate.

As used herein, “total copper” refers to the sum of all copper species in blood (for example, in serum or plasma). Total copper includes both ceruloplasmin (Cp)-bound copper and all species of non-ceruloplasmin bound copper. In general, total copper may be directly measured with high sensitivity and specificity by mass-spectroscopy, such as inductively coupled plasma-mass spectrometry (ICP-MS).

The term “NCC” refers to the fraction of total copper that is not bound to ceruloplasmin (i.e., “non-ceruloplasmin-bound copper”). Under commonly used estimation methods, NCC is estimated using direct measurements of total copper and Cp in the blood (such as, e.g., serum or plasma) and the following formula (Formula I):

$$N C C ( µM ) = \frac{P T C ( µg / L ) - ( 3.15 \times ceruloplasmin ( mg / L ) )}{63.5 ( µg / µmol )} .$$

The term “cNCC” refers to NCC as calculated using Formula I above. The calculation is premised on an assumption that six copper atoms are always bound to a single Cp molecule, and that NCC and ceruloplasmin concentrations are directly correlated. In reality, Cp may show considerable heterogeneity in the number of copper atoms associated per Cp molecule. This formula assumes that six copper atoms bind per one Cp molecule, but the copper/Cp ratio varies with disease state. In fact, 6-8 copper atoms can actually bind to Cp, and in WD usually fewer than six copper atoms are associated per Cp molecule.

In subjects treated with BC-TTM, non-ceruloplasmin-bound copper includes the fraction of total copper that is bound to albumin, transcuprein, and other less abundant plasma proteins (collectively referred to as labile-bound copper or LBC) or in tetrathiomolybdate-Cu-albumin tripartite complexes (TPCs). The concentration of TPCs cannot be directly measured, but in certain embodiments, the concentration of TPCs may be estimated using molybdenum concentration as a surrogate.

The term “NCC_{corrected}” refers to the fraction of total copper that is not bound to ceruloplasmin or in a TPC (i.e., LBC) and which is calculated by subtracting a direct measure of molybdenum in the blood (such as, e.g., serum or plasma) from the estimated NCC (or cNCC). “NCC_{corrected}” is thus a correction of the cNCC value to account for the presence of molybdenum-copper-albumin tripartite complexes in the blood of BC-TTM-treated subjects.

The term “dNCC” refers to NCC as directly measured using an NCC assay. For example, in certain embodiments, dNCC is directly measured using the NCC assay as disclosed in PCT Patent Application Publication No. WO2021/050850, filed on Sep. 11, 2020, which is incorporated by reference herein in its entirety.

The terms “LBC” or “labile-bound copper” refer to the fraction of total copper which is bound to albumin, transcuprein, and other less abundant plasma proteins. LBC thus comprises the fraction of total copper which is not bound to either ceruloplasmin or TPCs. In certain embodiments, the LBC fraction is directly measured using an LBC assay. For example, in certain embodiments, the LBC assay is as disclosed in PCT Patent Application Publication No. WO2021/050850, filed on Sep. 11, 2020, which is incorporated by reference herein in its entirety. In a biological sample in which no TPC is present, the NCC and the LBC fractions are the same.

The methods and uses of the disclosure are illustrated further by the following Example, which is not to be construed as limiting the disclosure in scope or spirit to the specific procedures and compounds described therein.

### Example 1: A Multicenter, Randomized, Controlled, Open-Label, Rater-Blinded Study to Evaluate Efficacy, Safety, Pharmacokinetics (PK), and Pharmacodynamics (PD) of Bis-Choline Tetrathiomolybdate (BC-TTM) Versus Standard of Care (SoC) in Pediatric Participants with Wilson Disease (WD)

### Overall Design

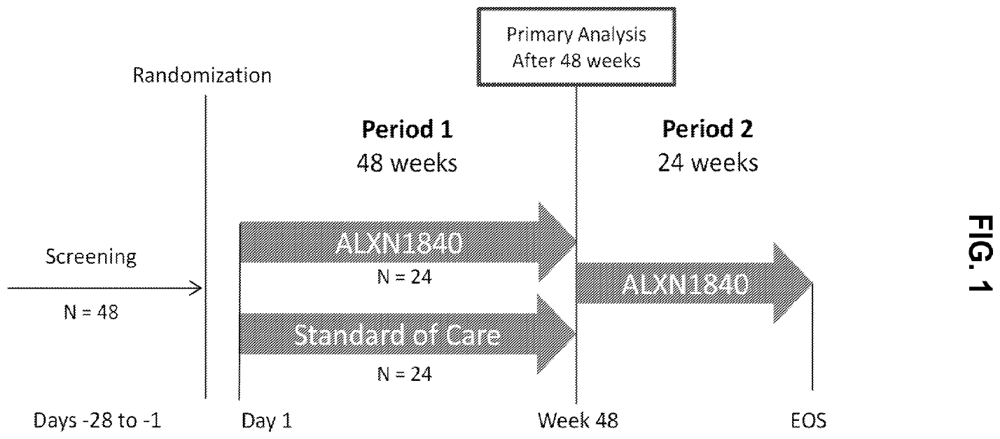

This is a randomized, controlled, open-label, rater-blinded study designed to assess the efficacy, safety, PK, and PD of BC-TTM versus SoC in pediatric participants aged 3 to <18 years with a confirmed diagnosis of WD, who meet pre-specified laboratory parameters and do not have decompensated cirrhosis. BC-TTM PK in plasma measured via total molybdenum and plasma ultrafiltrate (PUF) molybdenum, and PD measured by plasma total copper and LBC will be determined. FIG. 1 provides a schematic view of the study design.

The study includes 2 periods; the 48-week Period 1 serves to evaluate the effect of BC-TTM versus SoC on efficacy, safety, and PD. Participants who complete the 48-week Period 1 will be offered the opportunity to participate in a 24-week, open-label Period 2, i.e., up to 72 weeks in total, to evaluate the safety and efficacy of BC-TTM.

Approximately 48 participants will be randomized 1:1 to either BC-TTM or SoC treatment with the goal of obtaining 40 evaluable participants at Week 48. Participants in the Primary Evaluation Period will be stratified by age group (3 to <12 years, 12 to <18 years) and into 1 of 2 cohorts:
Cohort 1: Participants who have received SoC therapy (i.e., chelation therapy with penicillamine or trientine, treatment with zinc, or a combination of both chelation and zinc therapy) for >28 days prior to enrollment in the study.
Cohort 2: Participants who are treatment naïve or who have received SoC therapy for ≤28 days prior to enrollment in the study.

The primary enrollment and randomization objective is to have at least 12 participants in each age group and to achieve balanced treatment assignments both overall and within each age group. A secondary goal is to have at least 3 participants assigned to each treatment within each cohort of each age group.

To achieve this goal, participants will be randomized to treatment within one of the following 4 age groups/cohort strata:
Stratum 1:3 to <12 years old at randomization and Cohort 1 (prior SoC treatment >28 days) (a minimum of 6 participants)
Stratum 2: 3 to <12 years old at randomization and Cohort 2 (prior SoC treatment s 28 days) (a minimum of 6 participants)
Stratum 3: 12 to <18 years old at randomization and Cohort 1 (prior SoC treatment >28 days) (a minimum of 6 participants)
Stratum 4: 12 to <18 years old at randomization and Cohort 2 (prior SoC treatment s 28 days) (a minimum of 6 participants)

Throughout Period 1, participants randomized to receive BC-TTM will be administered BC-TTM orally daily at the following doses:
Participants aged 12 to <18 years will follow the same dosing paradigm as that in the ongoing Phase 3 Study-301 conducted in adults and adolescents. BC-TTM will be administered at a starting dose of 15 mg/day. Dose escalation is not permitted. Individualized doses ranging from 15 mg/day every other day to 15 mg/day are allowed. Doses of <15 mg every other day may be considered, with approval of the Alexion Medical Monitor.
For participants aged 3 to <12 years, a lower starting dose of 2.5 mg/day will be administered for at least 4 weeks based on scaling of the starting dose of 15 mg/day in the ongoing Study-301. Dose escalation is permitted but not required. The dose may be increased in increments of 2.5 mg daily with the permission of the Alexion Medical Monitor, depending on the participant's clinical status, NCC_{corrected} concentrations, and safety lab results. Dose increases must occur at least 4 weeks apart, and may only occur if no other dose modification (reduction or interruption) criteria apply. Participants who require doses of 15 mg daily may use the 15-mg adult tablet. The maximum dose for children under the age of 12 years is 15 mg/day.

Individualized BC-TTM dosing will be utilized throughout the study based on the following parameters:
Clinical criteria: dose-titration based on hepatic and neurological status
NCC_{corrected}: dose-titration based on NCC_{corrected} concentrations. The reference range for NCC_{corrected} is 0.8 to 2.3 μM.
Safety monitoring: dose modification criteria are based on regularly scheduled assessments for recognized hematological effects of copper lowering, hepatic testing, and neurological tests

Participants randomized to receive SoC treatment will continue their current therapy or initiate SoC, i.e., chelation therapy with penicillamine or trientine, zinc therapy, or a combination of both chelation and zinc therapy if they are not currently on SoC at the start of the study.

The study objectives and endpoints are presented in Table 1.

TABLE 1
Study Objectives and Endpoints

| | |
|---|---|
| Objectives | Endpoints |

Period 1: Up to Week 48
Primary

| | |
|---|---|
| To evaluate the efficacy of BC-TTM | Percentage change from baseline (Day 1) |
| administered for 48 weeks, compared | to 48 weeks in NCC in plasma. For BC- |
| to SoC, on copper control in | TTM-treated participants, the NCC in |
| participants with WD aged 3 to <18 | plasma will be corrected for the amount |
| years of age at the time of enrollment | of copper bound to the BC-TTM TPC |
| | (NCC_{corrected}) |

Secondary

| | |
|---|---|
| Evaluate the safety and tolerability of | Incidence of AEs/SAEs, AESIs, |
| BC-TTM administered for up to 48 | tolerability, clinical laboratory test data |
| weeks | (including liver function tests), |
| | neurological and physical examination |
| | findings, 12-lead ECG data, and vital |
| | signs. |
| Evaluate PD and biomarkers of BC- | AUEC for NCC |
| TTM vs SoC administered for 48 | AUEC for plasma total copper |
| weeks | Biomarkers: observed, absolute and |
| | percentage changes of ceruloplasmin- |
| | bound copper and ceruloplasmin |
| Evaluate the effects of BC-TTM and | NCC responder rate |
| SoC on the NCC responder rate | |
| Evaluate the effects of BC-TTM and | Change from baseline to Week 48 in the |
| SoC on participant reported disability | UWDRS Part II total score |
| status | |
| Evaluate the effects of BC-TTM and | Change from baseline in UWDRS Part III |
| SoC on rater-blinded neurological | total score or individual items/subscales, |
| status | as appropriate |
| Evaluate PK of BC-TTM administered | Estimation of PK parameters and |
| for 48 weeks | accumulation ratios |
| | PK: Cₘₐₓ, tₘₐₓ, C_{trough}, and AUCₜₐᵤ on Day |
| | 1, Day 43 (Week 6), and Day 337 (Week |
| | 48) for plasma total molybdenum and |
| | plasma ultrafiltrate molybdenum |
| | concentrations, accumulation ratio of Day |
| | 43 to Day 1 and Day 337 to Day 1 based |
| | on Cₘₐₓ, C_{trough}, and AUCₜₐᵤ |
| | Derived secondary PK parameters such |
| | as apparent total body clearance (CL/F) |
| | and apparent volume of distribution (V_{d}/F) |
| | (as appropriate) |
| Evaluate the effects of BC-TTM and | CGI-I |
| SoC on global clinical symptoms as | Change from Baseline to Week 48 in |
| assessed by the Investigator | CGI-S |
| Evaluate the effects of BC-TTM and | Change from Baseline to Week 48 in |
| SoC on hepatic status | MELD score (ages 12 years and older) or |
| | PELD score (ages 3 to <12 years) |
| | Change from Baseline to Week 48 in |
| | Modified Nazer score |

Exploratory

| | |
|---|---|
| Evaluate the effects of BC-TTM and | Change from Baseline to Week 48 in the |
| SoC on hepatic fibrosis | FIB-4 Index and by transient |
| | elastography |
| Evaluate the effects of BC-TTM and | Change from Baseline to Week 48 in |
| SoC on psychiatric symptoms | BPRS-24 and BPRS-C9 |
| Evaluate the effects of BC-TTM and | Change from Baseline to Week 48 in |
| SoC on QoL/PROs | QoL/PRO endpoint measures: |
| | EQ-5D or EQ-5DY |
| | PedsQL |
| | NOTE: These tests will be administered by |
| | parent/proxy for participants unable to |
| | complete independently. |
| Evaluate participant satisfaction of | Change from Baseline to Week 48 in TSQM-9 |
| treatment with BC-TTM and SoC | NOTE: This questionnaire will be |
| | administered by parent/proxy for participants |
| | unable to complete independently. |
| Evaluate the effects of BC-TTM and | Change from Baseline to Week 6 in 24- |
| SoC on 24-hour fecal copper and fecal Mo | hour fecal copper and fecal molybdenum |
| Evaluate the effects of BC-TTM and | Change from Baseline to Week 48 in 24- |
| SoC on 24-hour urinary copper and | hour urinary copper and urinary |
| urinary Mo | molybdenum |
| Explore other directly measured | Daily mean AUEC of NCC, and plasma |
| pharmacodynamics (PD) and | total copper from 0 to 24, and 24 to 48 weeks |
| biomarkers of BC-TTM | Observed, absolute and percent changes |
| | of copper levels (total copper, PUF |
| | copper, LBC) |
| Explore BC-TTM effect on initial | Time to first confirmed increase in plasma |
| decoppering phase compared to SoC | NCC and total copper concentration |
| based on directly measured PK/PD | Time to minimum and maximum concentration of: |
| and biomarkers | Plasma total copper |
| | Plasma NCC |
| | Plasma LBC |
| | Ratio plasma NCC:total copper |
| | Ratio plasma LBC:total copper |
| | Plasma ceruloplasmin |
| | Plasma CpC |
| | Ratio plasma ceruloplasmin:total copper |
| | Plasma CpC:total copper |
| | 24-hour urinary molybdenum |
| | Ratio urinary molybdenum:copper |
| | Ratio 24-hour urinary |
| | molybdenum:dosed molybdenum |
| Explore BC-TTM effect on subsequent | Time for return to pre-dose baseline for |
| maintenance phase compared to SoC | the following PK/PD parameters: |
| based on directly measured PK/PD | Plasma total copper |
| and biomarkers | Plasma NCC |
| | Plasma LBC |
| | Ratio plasma NCC: total copper |
| | Ratio plasma LBC:total copper |
| | Plasma ceruloplasmin concentration |
| | Plasma CpC concentration |
| | Ratio plasma ceruloplasmin:total copper |
| | Ratio plasma CpC:total copper |
| | 24-hour urinary molybdenum |
| | Ratio urinary molybdenum:copper |
| | Ratio urinary molybdenum:dosed |
| | molybdenum |

Period 2: Weeks 48 to 72
Exploratory

| | |
|---|---|
| Safety and tolerability of BC-TTM in | AEs/SAEs, AESI, tolerability, clinical |
| the Extension Period | laboratory test data, physical examination |
| | findings, vital signs, and 12-lead ECG |
| | data |
| | AUEC for NCC |
| Evaluate PD and biomarkers of BC- | AUEC for plasma total copper |
| TTM | Observed, absolute and percent changes |
| | of copper levels (total copper, PUF |
| | copper, LBC) |
| | Biomarkers: observed, absolute and |
| | percent changes of ceruloplasmin-bound |
| | copper and ceruloplasmin |
| Evaluate the effects of BC-TTM on | Change from Baseline in MELD/PELD |
| hepatic status | score and modified Nazer score |
| Evaluate the effects of BC-TTM on | Change from Baseline in UWDRS Part II |
| disability status | |
| Evaluate the effects of BC-TTM on | Change from Baseline in UWDRS Part III |
| neurological status | |
| Evaluate the effects of BC-TTM on | Change from Baseline in transient |
| hepatic fibrosis | elastography and FIB-4 index |
| Evaluate the effects of BC-TTM on | Change from Baseline in BPRS-24 and |
| psychiatric symptoms | BPRS-C9 |
| Evaluate the effects of BC-TTM on | Change from Baseline in CGI-I and CGI-S |
| global clinical symptoms as assessed | |
| by the Investigator | |
| Evaluate the effects of BC-TTM on | Change from Baseline to Week 72 in |
| QoL/PRO | QoL/PRO endpoint measures: |
| | EQ-5D or EQ-5D-Y |
| | PedsQL |
| | NOTE: These tests will be administered by |
| | parent/proxy for participants unable to |
| | complete independently. |
| Evaluate participant satisfaction of | Change from Baseline to Week 72 in |
| treatment with BC-TTM and SoC | TSQM-9 |
| | NOTE: This questionnaire will be |
| | administered by parent/proxy for participants |
| | unable to complete independently. |

Abbreviations:
AE = adverse event; AESI = adverse event of special interest; AUCtau = area under the plasma concentration versus time curve from time 0 to the end of the dosing interval; AUEC = area under the effect versus time curve; BPRS-24 = Brief Psychiatric Rating Scale-24; BPRS-C9 = Brief Psychiatric Rating Scale for children; CGI-I = Clinical Global Impression-Improvement Scale; CGI-S = Clinical Global Impression-Severity Scale; CL/F = apparent total body clearance; C ₘₐₓ = maximum observed concentration; CpC = ceruloplasmin-bound copper; C_{trough} = Trough (predose) concentration observed at the start of the dosing interval; ECG = electrocardiogram; EQ-5D(Y) = EuroQoL 5 Dimensions (Youth); FIB-4 = fibrosis-4; LBC = labile-bound copper; MELD = Model for End-stage Liver Disease; NCC = non-ceruloplasmin-bound copper; NCC_{corrected} = corrected NCC; PD = pharmacodynamics; PELD = Pediatric End-stage Liver Disease; PedsQL = Pediatric Quality of Life Inventory; PK = pharmacokinetics; PRO = patient-reported outcome; PUF = plasma ultrafiltrate; QoL = quality of life; SAE = serious adverse event; SoC = standard of care; tₘₐₓ = time to maximum concentration; TPC = tripartite complex; TSQM-9 = Treatment Satisfaction Questionnaire for Medication-9; UWDRS = Unified Wilson Disease Rating Scale; V_{d}/F = apparent volume of distribution.

### Scientific Rationale for Study Design

The study is designed as a randomized, open-label, exploratory study. The dosing strategy is similar to that employed in the current Phase 3 Study −301, with adjustments to the doses for pediatric patients based on the population PK simulations.

Following BC-TTM administration, the active drug moiety, i.e., the tetrathiomolybdate anion, rapidly binds copper to form TPCs, mostly in the liver and blood, and present as such in the systemic circulation. If TPCs are not rapidly formed, tetrathiomolybdate spontaneously undergoes serial hydrolysis to form molybdate, the most common form of nutrient molybdenum, and is excreted in the urine. The concentration of endogenous molybdenum in plasma is very low, therefore no background subtraction is required and plasma molybdenum can be attributed entirely to BC-TTM. Total molybdenum concentration cannot distinguish whether the molybdenum is protein-bound (mostly as TPC), free active drug as BC-TTM, intermediate hydrolysis products, or molybdate. To better characterize the amount of non-TPC-bound drug and its unbound degradation products, plasma PUF molybdenum has also been measured, which represents the free parent drug (BC-TTM), short-lived intermediate hydrolysis products, and molybdate, which may have originated from the tetrathiomolybdate or from food intake as a micronutrient. To better characterize the absorption, distribution, metabolism, and excretion of BC-TTM, the PK of both total molybdenum and PUF molybdenum will be characterized and described.

BC-TTM PK and PD in children and adolescents with WD will be assessed in this study. SoC PD will be assessed in children and adolescents. BC-TTM PK and PD parameters are expected to scale with the body weight per allometric rules since higher blood flow rates in children may have an impact on drug clearance. In addition, copper content within a participant's liver and, thus, liver volume may have an impact on the extent of drug binding in liver and clearance. Based on the BC-TTM mechanism of action, it is anticipated that drug-copper binding and chemical degradation do not differ in the pediatric population compared with adults, after taking into account body weight and liver volume factors.

Participants will be randomized after meeting all inclusion and none of the exclusion criteria. Participants will be randomized, stratified by cohort, via an interactive voice/web response system in a 1:1 ratio to treatment with BC-TTM or continued treatment with SoC in Cohort 1, or as continued or initial therapy in Cohort 2.

This study is rater-blinded for the UWDRS assessment only. The rater will be blinded and will have no knowledge of the participant's treatment assignment and no access to systems that could result in potential unblinding of treatment assignment. Both raters and participants will be instructed to avoid lines of inquiry, questions, and responses that could potentially lead to their unblinding. The rater assessments will be strictly limited to administration of the protocol specified instruments and assessments.

### Drug Dosing

Results from Studies 101, 102, 104, 106, 107, 108, 109, 201, and the ongoing Study 301 have shown that age, sex, or body weight are not significant covariates that may affect the PK of BC-TTM in adults and adolescents.

In the ongoing Phase 3 Study 301, participants were initially administered BC-TTM at a dose of 15 mg/day. Incremental dose increases are permitted, but not required, up to a maximum of 60 mg/day. In the completed 48-week Primary Evaluation Period of Study 301, the overall mean daily dose of BC-TTM was 15.6 mg with a minimum daily dose of 12.6 mg and a maximum daily dose of 19.8 mg. Results from the Primary Evaluation Period showed approximately 3-fold greater copper mobilization from tissue to blood during treatment with BC-TTM compared with SoC, as measured by daily mean dNCC AUEC_{0-48 weeks} (μmol/L).

Although 15 to 60 mg/day single or repeated daily doses have been shown to have acceptable safety profiles and to be generally well tolerated throughout the Phase 1 to Phase 3 clinical studies in both healthy adults and adult participants with WD, approximately 15% of the participants in Study 301 experienced Grade 2 ALT elevations during treatment with BC-TTM. In summary, the maximum daily dose permitted in this study (Study 302) has been set as 15 mg/day.

Selection of the starting and maximum doses for Study 302 in the pediatric population aged 3 to <12 years is based on a combination of standard body weight-based allometric principles and on the approach to account for potential changes in liver copper content in pediatric participants driven by age-based changes in liver volume.

Participants receiving the 1.25 mg mini-tablets who require a dose of 15 mg may be switched to the 15 mg tablet formulation. Participants may down-titrate to 15 mg every other day, or a lower or less frequent dose, as appropriate.

To support the dose selection, a population PK model for BC-TTM was developed based on data from healthy volunteers and patients with WD. Single dose PK data from 18 healthy volunteers (Study 102) and 27 patients (Study 201) were utilized for model building for the adult dosing. Simulations for pediatrics were based on typical parameter estimates without including variability components. For each simulation, the starting dose and concentration ranges were derived from the typical participants within the investigated age cohort at the extremes of the bodyweight range.

Based on the modelling results, additional pediatric PK/PD studies were deemed unnecessary as endorsed in the agreed pediatric investigational plan (PIP) with the EMA Decision P/0234/2020. The general approach of dose selection for pediatric investigation used here is consistent with the EMA guidance (ICH E11(R1), 2017).

Starting and maximal doses were selected using a conservative approach that involved consideration of lower end of the range, derived from the scaling methods, and further adjustment downward to a lower dose to ensure safety in the pediatric population aged 3 to <12 years. This cautious approach to starting and maximal dose selection (i.e., adjustment downward to the lower end of the projected dose range) for participants aged 3 to <12 years is supported by the fact that BC-TTM has greater binding affinity to copper compared with other treatments.

BC-TTM PK and PD processes are expected to scale with the body weight per allometric rules since higher blood flow rates in children may have an impact on drug clearance. In addition, copper content within a participant's liver and, thus, liver volume, may have an impact on extent of drug binding in liver and clearance. Based on the BC-TTM mechanism of action, it is expected that drug-copper binding and chemical degradation do not differ in the pediatric population compared with adults, after taking into account body weight and liver volume factors.

BC-TTM will be administered in the fasted state (1 hour before or 2 hours after meals) and will be taken with approximately 240 mL of water.

### Population Inclusion Criteria

Participants must be aged 3 to <18 years at time of signing the informed consent/assent. In addition, participants must have established diagnosis of WD by Leipzig-Score 4 documented by testing as outlined in the 2012 European Association for the Study of Liver WD Clinical Practice Guideline). Note: Historical test results for WD, including some or all of the following: presence of KF rings, neurologic symptoms, serum ceruloplasmin below the reference range, Coombs-negative hemolytic anemia, elevated liver or urinary copper, presence of mutations in the ATP7B gene, or other, as considered appropriate, may be used instead to confirm the diagnosis of Wilson disease. Participant's parent/proxy must be willing and able to give written informed consent and the participant must be willing to give written informed assent (if applicable as determined by the central or local Institutional Review Board [IRB]/Institutional [or Independent] Ethics Committee [lEC]). If allowable per local regulations, a participant's Legally Acceptable Representative (LAR) may provide informed consent if a participant is unable to do so. In addition, participants must have adequate venous access to allow collection of required blood samples and be able to swallow intact BC-TTM tablets or mini-tablets. Participants who require gastrostomy devices for feeding or medications may be enrolled if the inner diameter of the tube can accommodate an intact tablet or mini-tablet without obstruction. In addition, patients need to be willing to avoid intake of foods and drinks with high contents of copper throughout the study duration. Female participants of childbearing potential and male participants must follow protocol-specified contraception guidance. Capable of giving signed informed consent or assent, which includes compliance with the requirements and restrictions listed in the informed consent or assent form and in this protocol.

Participants are excluded from the study if any of the following criteria apply:
1. Decompensated hepatic cirrhosis.
2. MELD score >13 (ages 12 to <18) or PELD score >13 (ages 3 to <12).
3. Modified Nazer score >7.
4. Clinically significant gastrointestinal (GI) bleed within past 3 months.
5. Alanine aminotransferase (ALT) >2×upper limit of normal (ULN) for participants treated for >28 days with WD therapy (Cohort 1).
6. ALT >5×ULN for treatment naïve participants or participants who have been treated for ≤28 days (Cohort 2).
7. Marked neurological disease requiring either nasogastric feeding tube or intensive inpatient medical care.
8. Hemoglobin less than lower limit of the reference range for age and sex.
9. History of seizure activity within 6 months prior to informed consent/assent.
10. Previous use of BC-TTM or ammonium tetrathiomolybdate.
11. The use of an investigational drug within 30 days before initiation of the first dose of study intervention.
12. Participants in renal failure, defined as in end-stage renal disease on dialysis (chronic kidney disease stage 5 [CKD 5]) or estimated glomerular filtration rate <30 mL/min/1.73 m².
13. Active infection with hepatitis B virus (positive hepatitis B surface antigen) or C virus (participants with positive hepatitis C antibody result would require confirmation of active disease with a positive hepatitis C polymerase chain reaction test), or seropositivity for HIV.
14. Any disability acquired from trauma or another illness that, in the opinion of the Investigator, could interfere with evaluation of disability due to WD.
15. Systemic disease or other illness, or any deviation in laboratory values that are confirmed on re-examination to be clinically significant by the Investigator that would, in the opinion of the Investigator, compromise participant safety or interfere with the collection or interpretation of study results.
16. Pregnant (or females who are planning to become pregnant) or breastfeeding females.
17. Known sensitivity to BC-TTM, BC-TTM excipients (anhydrous di-calcium phosphate, anhydrous sodium carbonate), or any of the ingredients contained in BC-TTM or related compounds.
18. Regular alcohol consumption within 6 months prior to the study defined as >14 units for males or >7 units for females per week. One unit is equivalent to 14 g of alcohol: a half pint (approx. 240 mL) of beer, 1 glass (125 mL) of wine or 1 (25 mL) measure of spirits.
19. Abuse of illicit or prescribed drugs.
20. In the opinion of the Investigator, the participant and/or their parent/proxy is likely to be non-compliant or uncooperative during the study.

### Study Intervention

Study intervention is defined as any investigational intervention(s), marketed product(s), placebo, or medical device(s) intended to be administered to a study participant according to the study protocol.

Participants randomized to receive SoC treatment will continue their current therapy or initiate chelation therapy with penicillamine or trientine, zinc, or a combination of both chelation and zinc therapy if they are not currently on SoC at the start of the study. Standard of care treatment should be stored according to the details in the package labeling.

Details of BC-TTM administered in the study are provided in Table 2.

TABLE 2
Study Intervention Dosage and Mode of Administration

| | |
|---|---|
| Dose formulation | 15 mg white to off-white round enteric coated tablet |
| | 2.5 mg capsule containing 2 × 1.25 mg round white to off- |
| | white enteric coated mini-tablets |
| Unit dose strength(s) | 15 mg enteric coated tablet containing choline |
| | tetrathiomolybdate |
| | 1.25 mg enteric coated mini-tablet containing choline |
| | tetrathiomolybdate |
| Dosage level(s) | Participants aged 12 to <18 years: 15 mg/day |
| | Participants aged 3 to <12 years: starting dose of 2.5 |
| | mg/day, with increase in increments of 2.5 mg up to 15 |
| | mg/day, dose escalation requires the agreement of medical |
| | monitor |
| Route of administration | Oral |
| Use | Experimental/study intervention |
| IMP or NIMP | IMP |
| Packaging and labeling | BC-TTM will be provided in treatment kits that will each have a |
| | unique identification number and be packaged and labelled in |
| | accordance with all applicable regulatory requirements. At a |
| | minimum, the treatment kit label will provide the following |
| | information: Alexion study identification, batch number, |
| | directions for use, required storage conditions, caution |
| | statements (including “New Drug-Limited by Federal Law to |
| | Investigational Use” language), study identification, and expiry |
| | date. |

Abbreviations: IMP = investigational medicinal product; NIMP = non-investigational medicinal product.

### Prior and Concomitant Therapy

Any medication (including over-the-counter or prescription medicines, vitamins, and/or herbal supplements), or vaccine, or other specific categories of interest) that the participant is receiving within 30 days prior to enrollment or receives during the study must be recorded along with: reason for use, dates of administration including start and end dates, and dosage information including dose and frequency.

Medications specific for copper control in WD taken at any time prior to the study (i.e., all penicillamine, trientine or zinc ever taken for WD) will be recorded in a specific CRF for Prior WD Treatment.

### Allowed Medicine and Therapy

Investigators should use caution in the co-administration of drugs known to be substrates of cytochromes 2C9 and 2B6 (CYP2C9 and CYP2B6).

### Disallowed Medicine and Therapy

Concomitant use of penicillamine, trientine or zinc is prohibited for participants who are treated with BC-TTM during the randomized Primary Evaluation Period (Period 1) or Open-label Extension Period (Period 2). Standard of care medications may only be taken by participants randomized to SoC during Period 1. Use of nonprescription/over-the-counter medications, including herbal remedies, nutritional supplements, or mineral supplements containing copper, zinc, iron, or molybdenum after dosing on Day 1 through the end of the study is also disallowed. Vitamin E and estrogen should not be initiated during the study but can be continued if already being taken.

### Dose Modification

The BC-TTM dose should be lowered or interrupted if any of the relevant dose modification criteria are met. Deviation from the dose modification guidelines must be agreed with the Alexion Medical Monitor.

### Adolescent Participants

Adolescent participants (12 to <18 years) who are randomized to BC-TTM in Period 1 will be initiated on BC-TTM at 15 mg/day. Specific criteria for dose reduction or temporary interruption of dosing of BC-TTM are detailed in Table 3. Repeat testing of laboratory parameters which prompt dose modification criteria should follow the instructions in Table 3. Laboratory testing should be performed through the Central Laboratory if possible. Results from non-scheduled safety laboratory assessments performed at a local laboratory must be recorded in the CRF.

### Pediatric Participants

Pediatric participants (age 3 to <12 years) will be initiated on BC-TTM at 2.5 mg/day. Following the first 4 weeks, the dose may be increased, in increments of 2.5 mg at least 4 weeks apart to a maximum of 15 mg/day with the agreement of the Alexion Medical Monitor. Incremental dose increase is permitted but not required. Specific criteria for dose reduction, temporary interruption of dosing, or restriction of dose increases of BC-TTM are detailed in Table 4. Repeat testing of laboratory parameters that prompt dose modification criteria should follow the instructions in Table 4. Laboratory testing should be performed through the Central Laboratory if possible. Results from non-scheduled safety laboratory assessments performed by a local laboratory must be recorded in the CRF.

TABLE 3
BC-TTM Dose Modifications for Individual Adolescent Participants

| | | | Action with | Changes in Safety | |
|---|---|---|---|---|---|
| Test | Result | Conditions | BC-TTM Dosing | Monitoring^{a} | Rechallenge^{b, c} |
| ALT | >5 × ↑ | ALT above | Temporary interruption | Contact participant | At 15 mg QOD when |
| | from Baseline | reference | | within 48 hours to | ALT <2 × ↑ |
| | | range at | | arrange repeat testing | from Baseline. |
| | | Baseline | | (weekly repeat testing) | |
| | >5 × ULN | ALT within | Temporary interruption | Contact participant | At 15 mg QOD when |
| | | reference | | within 48 hours to | ALT <2 × ULN. |
| | | range at | | arrange repeat testing | |
| | | Baseline | | (weekly repeat testing) | |
| | >2 × ↑ | ALT above | Reduce dose to 15 mg QOD | Weekly repeat testing | Not applicable. |
| | from Baseline | reference | if on 15 mg QD. No further | | |
| | | range at | dose ↑ until resolution | | |
| | | Baseline | of abnormality. | | |
| | >2 × ULN | ALT within | Reduce dose to 15 mg QOD | Weekly repeat testing | Not applicable. |
| | | reference | if on 15 mg QD. No further | | |
| | | range at | dose ↑ until resolution | | |
| | | Baseline | of abnormality. | | |
| Increased | >300 to 500 mg/dL | None | Reduce dose to 15 mg | Weekly repeat testing | Not applicable |
| triglycerides | or | | QOD if on 15 mg QD. | | |
| | >3.4 to 5.6 mmol/L | | | | |
| | >500 mg/dL | | Temporary interruption | | At 15 mg QOD |
| | or | | | | when triglyceride |
| | 5.6 mmol/L | | | | concentrations |
| | | | | | return to baseline |
| Increased | >300 to 400 mg/dL | None | Reduce dose to 15 mg | Weekly repeat testing | Not applicable |
| total | or | | QOD if on 15 mg QD. | | |
| cholesterol | >7.8 to 10.3 mmol/L | | | | |
| | >400 mg/dL | | Temporary interruption | | At 15 mg QOD |
| | or | | | | when cholesterol |
| | >10.3 mmol/L | | | | concentrations |
| | | | | | return to baseline |
| Hemoglobin | <8 g/dL in | None | Temporary interruption | Weekly repeat testing | At 15 mg QOD when |
| | the absence | | | | hemoglobin and |
| | of bleeding | | | | other hematology |
| | | | | | parameters |
| | | | | | (neutrophils |
| | | | | | and platelets) |
| | | | | | are at baseline |
| | | | | | concentration. |
| | >30% ↓ | None | Reduce dose to 15 mg QOD | Weekly repeat testing | Not applicable. |
| | from Baseline | | if on 15 mg QD. No further | | |
| | | | dose ↑ until resolution | | |
| | | | of abnormality. | | |
| Platelets | <30,000 μL | None | Temporary interruption | Weekly repeat testing | At 15 mg QOD when |
| | | | | | platelets and |
| | | | | | other hematology |
| | | | | | parameters |
| | | | | | (neutrophils and |
| | | | | | hemoglobin) are |
| | | | | | at baseline |
| | | | | | concentration. |
| | >30% ↓ | Platelets below | Reduce dose to 15 mg QOD | Weekly repeat testing | Not applicable. |
| | from Baseline | reference range | if on 15 mg QD. No further | | |
| | | at Baseline | dose ↑ until resolution | | |
| | | | of abnormality. | | |
| Neutrophils | <1.0 × 10³/μL | None | Temporary interruption | Weekly repeat testing | At 15 mg QOD when |
| | | | | | neutrophils and |
| | | | | | other hematology |
| | | | | | parameters |
| | | | | | (hemoglobin and |
| | | | | | platelets) are |
| | | | | | at Baseline |
| | | | | | concentration. |
| | >30% ↓ | Neutrophils | Reduce dose to 15 mg QOD | Weekly repeat testing | Not applicable. |
| | from Baseline | below reference | if on 15 mg QD. No further | | |
| | | range at | dose ↑ until resolution | | |
| | | Baseline | of abnormality. | | |
| Bilirubin | >2 × ULN | Accompanied by | Temporary interruption | Weekly repeat testing | At 15 mg QOD |
| | | ALT >3 × ULN, | | | or less frequent, |
| | | indicative of | | | when bilirubin |
| | | liver injury | | | is below ULN. |
| | | | | | Rechallenge |
| | | | | | under these |
| | | | | | conditions |
| | | | | | requires |
| | | | | | approval of |
| | | | | | the Alexion |
| | | | | | Medical Monitor. |

| | | | | |
|---|---|---|---|---|
| Neurological | Evidence of neurologic worsening | Investigator and Alexion Medical | All neurologic worsening | Discuss with |
| assessment | by AEs or by neurologic physical | Monitor will evaluate the need for | should be documented as | the Alexion |
| | exam assessment. | dose modification (interruption, | AEs and followed up until | Medical Monitor. |
| | | increase or decrease) based on | study completion or | |
| | | copper control parameters and | resolution of symptoms. | |
| | | relevant clinical data. Rationale | | |
| | | for dosing decision must be | | |
| | | documented in study record and | | |
| | | reevaluated at the next study visit. | | |
| Psychiatric | Evidence of clinically significant | Investigator and Alexion Medical | Worsening psychiatric | Discuss with |
| assessment | acute psychiatric include, but is | Monitor will evaluate the need for | symptoms will be | the Alexion |
| | not limited to, suicidality, acute | dose modification (interruption, | documented as AEs in the | Medical Monitor. |
| | depression, or psychosis. | increase or decrease) based on | eCRF and will be followed | |
| | | copper control parameters and | until completion of the | |
| | | relevant clinical data. Rationale | study or resolution of | |
| | | for dosing decision must be | symptoms. | |
| | | documented in study record and | | |
| | | reevaluated at the next study visit. | | |

^{a}For changes scheduled during this time period.
^{b}For rechallenges, participants who were on BC-TTM 15 mg QOD should be rechallenged at the 15 mg QOD dose.
^{c}The Investigator, in consultation with the Medical Monitor, may change dose and dose frequency in participants who require rechallenge.
Abbreviations: AEs = adverse event; ALT = alanine aminotransferase; eCRF = electronic case report form; QD = once daily; QOD = every other day; ULN = upper limit of normal.

TABLE 4
BC-TTM Dose Modifications for Individual Pediatric Participants

| | | | Action with | Changes in Safety | |
|---|---|---|---|---|---|
| Test | Result | Conditions | BC-TTM Dosing | Monitoring^{a} | Rechallenge^{b, c} |
| ALT | >5 × ↑ | ALT above | Temporary interruption | Contact participant | At 1.25 mg QD when |
| | from Baseline | reference | | within 48 hours to | ALT <2 × ↑ |
| | | range at | | arrange repeat testing | from Baseline. |
| | | Baseline | | (weekly repeat testing) | |
| | >5 × ULN | ALT within | Temporary interruption | Contact participant | At 1.25 mg QD when |
| | | reference | | within 48 hours to | ALT <2 × ULN. |
| | | range at | | arrange repeat testing | |
| | | Baseline | | (weekly repeat testing) | |
| | >2 × ↑ | ALT above | Reduce dose to previous | Weekly repeat testing | Not applicable. |
| | from Baseline | reference | dose level if up-titration | | |
| | | range at | has occurred or reduce | | |
| | | Baseline | dose to 1.25 mg QD if on | | |
| | | | 2.5 mg QD. No further | | |
| | | | dose ↑ until resolution | | |
| | | | of abnormality. | | |
| | >2 × ULN | ALT within | Reduce dose to previous | Weekly repeat testing | Not applicable. |
| | | reference | dose level if up-titration | | |
| | | range at | has occurred or reduce | | |
| | | Baseline | dose to 1.25 mg QD if on | | |
| | | | 2.5 mg QD. No further | | |
| | | | dose ↑ until resolution | | |
| | | | of abnormality. | | |
| Increased | >300 to 500 mg/dL | None | Reduce dose to previous | Weekly repeat testing | Not applicable |
| triglycerides | or | | dose level if up-titration | | |
| | >3.4 to 5.6 mmol/L | | has occurred or reduce | | |
| | | | dose to 1.25 mg QD if on | | |
| | | | 2.5 mg QD. No further dose | | |
| | | | increase until resolution | | |
| | | | of abnormality. | | |
| | >500 mg/dL | | Temporary interruption | | At 1.25 mg QD |
| | or | | | | when triglyceride |
| | 5.6 mmol/L | | | | concentrations |
| | | | | | return to baseline |
| Increased | >300 to 400 mg/dL | None | Reduce dose to 1.25 mg | Weekly repeat testing | Not applicable |
| total | or | | QD if on 2.5 mg QD. | | |
| cholesterol | >7.8 to 10.3 mmol/L | | | | |
| | >400 mg/dL | | Temporary interruption | | At 1.25 mg QOD |
| | or | | | | when cholesterol |
| | >10.3 mmol/L | | | | concentrations |
| | | | | | return to baseline |
| Hemoglobin | <8 g/dL in | None | Temporary interruption | Weekly repeat testing | At 1.25 mg QD |
| | the absence | | | | when hemoglobin |
| | of bleeding | | | | and other hematology |
| | | | | | parameters |
| | | | | | (neutrophils and |
| | | | | | platelets) are |
| | | | | | at Baseline |
| | | | | | concentration. |
| | >30% ↓ | None | Reduce dose to previous | Weekly repeat testing | Not applicable. |
| | from baseline | | dose level if up-titration | | |
| | | | has occurred or reduce dose | | |
| | | | to 1.25 mg QD if on 2.5 mg | | |
| | | | QD. No further dose ↑ until | | |
| | | | resolution of abnormality. | | |
| Platelets | <30,000 μL | None | Temporary interruption | Weekly repeat testing | At 1.25 mg QD |
| | | | | | when platelets and |
| | | | | | other hematology |
| | | | | | parameters |
| | | | | | (neutrophils and |
| | | | | | hemoglobin) are |
| | | | | | at Baseline |
| | | | | | concentration. |
| | >30% ↓ | Platelets | Reduce dose to previous | Weekly repeat testing | Not applicable. |
| | from Baseline | below reference | dose level if up-titration | | |
| | | range at | has occurred or reduce dose | | |
| | | Baseline | to 1.25 mg QD if on 2.5 mg | | |
| | | | QD. No further dose ↑ until | | |
| | | | resolution of abnormality. | | |
| Neutrophils | <1.0 × 10³/μL | None | Temporary interruption | Weekly repeat testing | At 1.25 mg QD when |
| | | | | | neutrophils and |
| | | | | | other hematology |
| | | | | | parameters |
| | | | | | (hemoglobin and |
| | | | | | platelets) are |
| | | | | | at Baseline |
| | | | | | concentration. |
| | >30% ↓ | Neutrophils | Reduce dose to previous dose | Weekly repeat testing | Not applicable. |
| | from Baseline | below reference | level if up-titration has | | |
| | | range at | occurred or reduce dose to | | |
| | | Baseline | 1.25 mg QD if on 2.5 mg QD. | | |
| | | | No further dose ↑ until | | |
| | | | resolution of abnormality. | | |
| Bilirubin | >2 × ULN | Accompanied by | Temporary interruption | Weekly repeat testing | At 1.25 mg QD or |
| | | ALT >3 × ULN, | | | less frequent, |
| | | indicative of | | | when bilirubin |
| | | liver injury | | | is below ULN. |
| | | | | | Rechallenge under |
| | | | | | these conditions |
| | | | | | requires approval |
| | | | | | of the Alexion |
| | | | | | Medical Monitor. |

| | | | | |
|---|---|---|---|---|
| Neurological | Evidence of neurologic worsening by | Investigator and Alexion | All neurologic worsening | Discuss with |
| assessment | AEs or by neurologic physical exam | Medical Monitor will evaluate | should be documented as | the Alexion |
| | assessment. | the need for dose modification | AEs and followed up until | Medical Monitor. |
| | | (interruption, increase or | study completion or | |
| | | decrease) based on copper | resolution of symptoms. | |
| | | control parameters and relevant | | |
| | | clinical data. Rationale for | | |
| | | dosing decision must be | | |
| | | documented in study record and | | |
| | | re-evaluated at the next study | | |
| | | visit. | | |
| Psychiatric | Evidence of clinically significant | Investigator and Alexion | Worsening psychiatric | Discuss with |
| assessment | acute psychiatric worsening which | Medical Monitor will evaluate | symptoms will be | the Alexion |
| | may include, but is not limited to, | the need for dose modification | documented as AEs in | Medical Monitor. |
| | suicidality, acute depression, or psychosis. | (interruption, increase or | the eCRF and will be | |
| | | decrease) based on copper control | followed until completion | |
| | | parameters and relevant clinical | of the study or resolution | |
| | | data. Rationale for dosing | of symptoms. | |
| | | decision must be documented in | | |
| | | study record and re-evaluated at | | |
| | | the next study visit. | | |

^{a}For changes in safety monitoring, weekly repeat testing for laboratory parameters can be completed by a home healthcare nurse if a routine study visit is not scheduled during this time period.
^{b}For rechallenges, participants who were on BC-TTM 2.5 mg QD should be rechallenged at the 1.25 mg QD dose.
^{c}The Investigator, in consultation with the Medical Monitor, may change dose and dose frequency in participants who require rechallenge.
Abbreviations: AEs = adverse event; ALT = alanine aminotransferase; eCRF = electronic case report form; QD = once daily; QOD = every other day; ULN = upper limit of normal.

Intervention after the End of the Study: Following completion of Period 2 of the study, participants will either: transition to therapy that was discontinued before enrollment, or participants who have successfully completed all study assessments and were not withdrawn prematurely may be eligible for post-study access if deemed in the best interest of the patient by the treating physician. Participants will receive study drug for up to 2 years or until 1) the study drug is registered or approved and available by prescription or 2) the study drug can be provided via Alexion post-study/early access programs as allowed by local laws and regulations, whichever occurs first. Only the investigational drug will be available for post-study/early access. All participants should return to the site for the EOS Visit on Day 197 of Period 2 (+/−7 days).

### Discontinuation of Study Intervention

In rare instances, it may be necessary for a participant to permanently discontinue (definitive discontinuation) the study intervention. If the study intervention is definitively discontinued, the participant should have an Early Termination Visit and return within 4 weeks to be evaluated for safety follow-up.

Participants must be considered for discontinuation from study intervention if any of the following occur during the study:
Occurrence of a decompensation cirrhosis event that is not responsive to treatment
A decompensation cirrhosis event is defined as acute esophageal or gastric variceal bleeding, development of new overt hepatic encephalopathy, or substantive de novo ascites formation
Serious hypersensitivity reaction
Severe uncontrolled infection
Use of disallowed medication
Pregnancy or planned pregnancy; or
Alexion or the Investigator deems it is necessary for the participant.

### Study Assessments and Procedures

Assessment of Copper: Because measures of non-ceruloplasmin-bound copper control in the blood are the primary assessment for efficacy of treatment with BC-TTM, plasma samples will be collected to measure total copper, ceruloplasmin, ceruloplasmin-bound copper (CpC), NCC, and LBC in the blood. In BC-TTM-treated patients, an additional calculation will be performed that corrects the NCC level for the amount of copper bound to the BC-TTM tripartite complex (NCC_{corrected}).

The AUEC for plasma total copper concentration over time aims to quantify the dynamic tissue mobilization and decoppering effect of BC-TTM. This assessment is also applicable to SoC treatments. AUEC will be calculated for direct NCC and plasma total copper.

Ongoing bioanalytical method development of new techniques to measure directly the TPC copper may allow additional analyses of copper control to be performed. As the method to indirectly estimate NCC concentration results in approximately 20% of samples from healthy participants yielding physiologically impossible negative values for NCC, the direct NCC and LBC assays have been developed. The LBC method measures exchangeable plasma copper that is not bound to either ceruloplasmin or the BC-TTM TPC.

Model for End-stage Liver Disease and Pediatric End-stage Liver Disease scores: The Model for End-stage Liver Disease (MELD) is a scoring system for assessing the severity of chronic liver disease in adults and adolescents aged 12 years and above. The MELD score (range 6-40, with higher values indicating more advanced disease) uses the participant's values for serum bilirubin, serum creatinine, and the international normalized ratio (INR) for prothrombin time to predict survival. In participants with a MELD score >11, the serum sodium is also taken into account.

The Pediatric End-stage Liver Disease (PELD) score is used to estimate 90-day survival in the absence of liver transplantation. The components of the PELD score are total bilirubin, INR, albumin, age, and growth failure. The PELD cutoff of >13 was chosen to exclude participants with advanced liver failure, comparable to a MELD score >13 or a modified Nazer score >7.

Unified Wilson Disease Rating Scale (Parts I, II, and Ill): The UWDRS is a clinical rating scale designed to evaluate the neurological manifestations of WD that generally can be divided into 3 movement disorder syndromes: dystonic, ataxic, and Parkinsonian syndrome. The UWDRS comprises 3 parts: UWDRS Part I (level of consciousness, item 1), UWDRS Part II (a participant-reported review of daily activity items [disability], items 2 to 11), and UWDRS Part Ill (a detailed neurological examination, items 12 to 34).

The UWDRS Part I and Part Ill will be assessed by a neurologist who is blinded to the treatment randomization, while UWDRS Part II may be reported to a non-blinded member of the study team by the participant, family member, or caregiver. The UWDRS has not been formally evaluated in children. However, the components of Part I (level of consciousness), Part II (participant or caregiver-reported disability) and Part Ill (neurologic examination findings) are not fundamentally different between adults and children. Participants aged 12 years and older are expected to be able to comply with UWDRS assessments without modification. The UWDRS assessments should be conducted to the greatest extent feasible in children <12 years.

Clinical Global Impression-Severity Scale and the Clinical Global Impression-Improvement Scale: The Clinical Global Impression (CGI) rating scales are commonly used measures of symptom severity, treatment response, and the efficacy of treatments in treatment studies of adult and pediatric participants with mental disorders.

Clinical Global Impression-Severity Scale: The Clinical Global Impression-Severity scale (CGI-S) is a 7-point scale that requires the clinician to rate the severity of the participant's illness at the time of assessment, relative to the clinician's past experience with participants who have the same diagnosis. Considering total clinical experience, a participant is assessed on severity of illness at the time of rating as: 1, normal, not at all ill; 2, borderline ill; 3, mildly ill; 4, moderately ill; 5, markedly ill; 6, severely ill; or 7, extremely ill.

Clinical Global Impression-Improvement Scale: The Clinical Global Impression-Improvement scale (CGI-1) is a 7-point scale that requires the clinician to assess how much the participant's illness has improved or worsened relative to a baseline state at the beginning of the intervention and rated as: 1, very much improved; 2, much improved; 3, minimally improved; 4, no change; 5, minimally worse; 6, much worse; or 7, very much worse.

Fibrosis-4 Index and Transient Elastography: The FIB-4 Index is a formula used to predict liver fibrosis based on standard biochemical values (ALT, AST, and platelet count) and age. The FIB-4 Index will be calculated by a Central Laboratory. Transient elastography is a non-invasive imaging method that evaluates the degree of liver fibrosis or fatty deposits in the liver, by determining the speed of sound waves through the liver utilizing a sonogram.

Modified Nazer Score: The modified Nazer score is an assessment of liver status and consists of a composite of 5 laboratory parameters: AST, INR, bilirubin, albumin, and white blood cell count. The score has a total range of 0 to 20, and lower values indicate a healthier liver status.

Brief Psychiatric Rating Scale-24: The Brief Psychiatric Rating Scale-24 (BPRS-24) is a 24-item instrument for adolescents aged 12 to <18 years that allows the rater to measure psychopathology severity. The presence and severity of psychiatric symptoms are rated on a Likert scale ranging from 1 (not present) to 7 (extremely severe). The BPRS-24 can be performed by a qualified person (e.g., neurologist, psychiatrist, psychologist, licensed mental health practitioner, social worker, etc.) who has completed the training required to administer the instrument. The BPRS-C9 is a 9-item instrument for children aged 3 to <12 years. The presence and severity of symptoms are rated on a scale ranging from 1 (not present) to 6 (extremely severe). As with the BPRS-24, the BPRS-C9 can be performed by a qualified person who has been appropriately trained.

EuroQoL 5 Dimensions: The EuroQoL 5 Dimensions (EQ-5D) consists of 2 different assessments—the EQ-5D-5L Descriptive System and the EQ Visual Analogue Scale (VAS). The descriptive system comprises measures of health-related quality of life state and consists of 5 dimensions: mobility, self-care, usual activities, pain/discomfort, and anxiety/depression. Each dimension has 5 levels of severity: no problems, slight problems, moderate problems, severe problems, or extreme problems. The EQ VAS records the participant's self-rated health on a vertical VAS. Together, this can be used as a quantitative measure of health outcome that reflects the participant's own judgement. Note that although the EQ-5D is designed for use in participants aged 16 years and older, the EQ-5D can also be used in participants aged 12 to 15 years and is appropriate given these participants will be followed for several years in the study.

EQ-SD Youth: The child-friendly EQ-5D version (EQ-5D-Y) was introduced as a more comprehensible instrument suitable for children and adolescents. The wording was changed to be more suitable for children and adolescents, the most severe label for the mobility dimension was changed from “confined to bed” to “a lot of problems walking about” to increase the applicability and sensitivity of the mobility dimension, and the instructions for the EQ VAS task were simplified, making the task easier to complete and to score (https://euroqol.org/eq-5d-instruments/eq-5d-y-about/).

The Treatment Satisfaction Questionnaire for Medication-9 (TSQM-9) is used to assess the overall level of satisfaction or dissatisfaction with medication participants are taking. This composite scale is comprised of 3 items on the TSQM-9 survey:
Overall, how confident are you that taking this medication is a good thing for you?
How satisfied are you that good things about this medication outweigh the bad things?
Taking all things into account, how satisfied or dissatisfied are you with this medication?

The TSQM-9 is expected to be completed by adolescent participants and by the parent or caregiver for younger participants.

The Pediatric Quality of Life Inventory (PedsQL™) Measurement Model is a modular approach to measuring health-related quality of life in healthy children and adolescents and those with acute and chronic health conditions. The 23-item PedsQL™ Generic Core Scales were designed to measure the core dimensions of health as delineated by the World Health Organization (WHO), as well as role (school) functioning. The 4 Multidimensional Scales and 3 Summary Scores are:

### Scales

Physical Functioning (8 items)
Emotional Functioning (5 items)
Social Functioning (5 items)
School Functioning (5 items)

### Summary Scores

Total Scale Score (23 items)
Physical Health Summary Score (8 items)
Psychosocial Health Summary Score (15 items)

Urinary and Fecal Copper Excretion: 24-hour urinary copper excretion will be assessed at Baseline, Week 6, Week 24, and Week 48 in all participants. 24-hour fecal copper excretion will be assessed at Baseline and Week 6 in all participants. This assessment is optional.

Physical Examinations: A complete physical examination will include, at a minimum, assessments of the cardiovascular, respiratory, GI, and neurological systems. An abbreviated physical examination consists of a body system relevant examination based upon Investigator judgment and participant symptoms. At least 1 body system must be checked for an abbreviated examination. A symptom-driven physical examination may be performed at other times, at the Principal Investigator's discretion.

Adverse Events of Special Interest: Any new neurological symptom or clinically significant worsening of an ongoing neurological symptom after initiation of study intervention (BC-TTM or SoC) will be designated an adverse event of special interest (AESI), whether serious or non-serious. If a participant has an AESI, in addition to assessments deemed clinically relevant by the Investigator, the following assessments should be performed to the extent possible to help assess the AE and participant status: UWDRS Part Ill, and the CGI-I and CGI-S. The Investigator or Sub Investigator can perform additional assessments or laboratory testing at their discretion. SAEs of special interest will be assessed by a panel of 3 independent neurologists not participating in the study. The panel will assess the probability that clinically significant worsening or a new clinically significant neurological symptom is related to disease progression or caused by the study intervention (BC-TTM or SoC). They will be blinded to the treatment given to the participant. All available relevant participant information will be provided to this panel to aid in their assessment.

### Pharmacokinetics:

Whole blood samples will be collected for analyzing plasma concentrations of total molybdenum and PUF molybdenum as specified in the Schedule of Activities (SoA).
Instructions for the collection and handling of biological samples will be provided by Alexion. The actual date and time (24-hour clock time) of each sample will be recorded.
Excess/additional samples may be stored for up to 5 years and used for PD and/or diagnostic biomarker development and research to understand the pathways associated with the mechanism of action of BC-TTM. These samples will not be used for genetic analyses (i.e., RNA or DNA analyses).
Genetic analyses will not be performed on the whole blood samples collected for PK/PD analysis.

### Pharmacodynamics:

Whole blood will be collected for measurement of plasma total and PUF copper, NCC, and LBC as specified in the SoA.
Instructions for the collection and handling of biological samples will be provided by Alexion. The actual date and time (24-hour clock time) of each sample will be recorded.
Plasma samples will be used to evaluate BC-TTM or SoC PD via the measurement of total copper and copper measured as NCC and PUF copper, and/or LBC, or assessed via NCC/NCC_{corrected} methods. Samples collected for PD measurements of BC-TTM and SoC may also be used to evaluate safety or efficacy aspects related to concerns arising during or after the study.
Excess/additional samples may be stored for up to 5 years and used for PD and/or diagnostic biomarker development and research to understand the pathways associated with the mechanism of action of BC-TTM. These samples will not be used for genetic analyses (i.e., RNA or DNA analyses).

Genetics: A blood sample for DNA isolation will be collected from participants who have consented to participate in the genetic analysis component of the study. Participation is optional. Participants who do not wish to participate in the genetic research may still participate in the study.
Genetic variation in the ATP7B gene may impact a patient's response to study intervention, susceptibility to, and severity and progression of disease. Therefore, where local regulations and IRB/IEC allow, a blood sample will be collected for DNA analysis from consenting patients.
DNA samples will be used for research related to WD. They may also be used to develop tests/assays including diagnostic tests related to BC-TTM and WD
DNA samples will be analyzed for variants in the coding and regulatory sequences of the ATP7B gene and other genes, if considered related to WD. Additional analyses may be conducted if it is hypothesized that this may help further understand the clinical data, such as identifying gene mutations that may affect the metabolism of BC-TTM.
The samples may be analyzed as part of a multi-study assessment of genetic factors involved in the response to BC-TTM or study interventions of this class to understand study disease or related conditions.

### Efficacy Analyses

The primary endpoint, the percentage change from Baseline to 48 weeks in NCC/NCC_{corrected} concentrations, will be analyzed by treatment group within each cohort and by treatment group overall using descriptive summary statistics. Analyses will be based on the Per Protocol Set and Full Analysis Set. BC-TTM biomarkers such as ceruloplasmin and CpC will be measured in plasma/serum samples.

Analyses of secondary efficacy endpoints will be based on the Full Analysis Set. All secondary efficacy endpoints will be summarized by treatment groups within each cohort and by treatment group overall using descriptive statistics.

All exploratory endpoints will be summarized by treatment groups within each cohort and by treatment group overall using descriptive statistics using the Full Analysis Set.

All safety analyses will be performed on the Safety Set. Safety analyses will include all AEs, ECGs, clinical laboratory data, physical examinations, and vital sign measurements using descriptive statistics. An AE is any untoward medical occurrence in a patient or clinical investigation participant administered a pharmaceutical product and which does not necessarily have to have a causal relationship with this treatment (ICH E2A). Note: An AE can therefore be any unfavorable and unintended sign (including an abnormal laboratory finding), symptom, or disease (new or exacerbated) temporally associated with the use of study intervention.

If an event is not an AE per definition above, then it cannot be an SAE even if serious conditions are met (e.g., hospitalization for signs/symptoms of the disease under study, death due to progression of disease). An SAE is defined as any untoward medical occurrence that, at any dose: results in death, is life-threatening; requires inpatient hospitalization or prolongation of existing hospitalization, results in persistent disability/incapacity, is a congenital anomaly/birth defect, and other situations.

Treatment-emergent adverse events (TEAEs) are defined as those AEs with onset after the first dose of randomized treatment or existing events that worsened in severity after the first dose of randomized treatment. Events reported with a partial onset date (e.g., month and year are reported, but the day is missing) will be considered to be treatment-emergent if it cannot be confirmed that the event onset was prior to the first dose of study drug based on the available date entries.

Additionally, for patients randomized to SoC in Period 1 who switch to BC-TTM in Period 2, any AEs that initiate after the switch or existing events that worsen in severity will be attributed to BC-TTM.

An overall summary of TEAEs will be presented by treatment, including frequency of participants experiencing the event (n) and relative frequency (n/N*100, where N is the number of patients in the Safety Set for each treatment group). The summary will include categories indicating how many events are TEAEs, treatment-emergent SAEs, and treatment-emergent non-SAEs. Within TEAEs, the following subcategories will also be summarized:
Severity of TEAEs (Grade 1 through Grade 5)
Related TEAEs (not related, related)
TEAEs leading to withdrawal of study drug
TEAEs leading to death

The incidence of AEs and SAEs will be summarized by System Organ Class and Preferred Term for each treatment and overall, and by relationship to study intervention. Adverse events will also be summarized by treatment and overall by severity. Serious AEs and AEs resulting in withdrawal from the study will be listed. Participants having multiple AEs within a category (e.g., overall, System Organ Class, Preferred Term) will be counted once in that category. For severity tables, a participant's most severe event within a category will be counted.

Changes from Baseline in vital sign measurements and laboratory assessments (e.g., chemistry, hematology, coagulation, and urinalysis) will be summarized by treatment. Laboratory parameter values will be graded according to the National Cancer Institute Common Terminology Criteria for Adverse Events (CTCAE). Shift tables by treatment will be produced for these laboratory parameters. These tables will summarize the number of participants with each baseline grade relative to the reference ranges and changes to the worst highest grade assessed post dose during the study.

Electrocardiogram parameters will be measured at the specified time points as per the SoA, including heart rate, PR, RR, QRS, QT, and QT intervals corrected for heart rate using Fridericia's formula (QTcF intervals). The average of the triplicate ECG readings at the time points collected will be calculated, and changes from Baseline values will be assessed by each treatment.

### Pharmacokinetic (PK), Pharmacodynamic (PD), and Biomarker Analyses

For PK, PD, and biomarker endpoints, analyses will be performed using the PK/PD Analysis Set.

The following plasma PK parameters will be calculated for total molybdenum and PUF molybdenum, if measured, using noncompartmental methods with Phoenix® WinNonlin® (Certara USA Inc., Princeton, New Jersey) Version 8.0 or higher or SAS Version 9.3 or higher (SAS Institute Inc., Cary, North Carolina), as applicable. Calculations will be based on the actual sampling times recorded during the study.
Maximum observed concentration (Cₘₐₓ)
Time to maximum concentration (Tₘₐₓ)
Trough (predose) concentration observed at the start of the dosing interval (C_{trough})
Area under the plasma concentration versus time curve (AUC) over the dosing interval (AUCₜₐᵤ)

Additional plasma PK parameters may be calculated if deemed appropriate. Population PK analysis may be formed with pooled data from other clinical studies if deemed appropriate. Population PK parameters such as, but may not be limited to, apparent total body clearance (CL/F) and apparent volume of distribution (V_{d}/F) will be estimated for addition modeling and simulation purposes.

Plasma concentrations of total molybdenum and PUF molybdenum (if measured) versus time data will be presented in a data listing by participant. Plasma concentration data will be summarized separately by analyte and time point for each treatment by day using the following descriptive statistics: number of participants, arithmetic mean, geometric mean (GM), SD, coefficient of variation (CV), GMCV, median, minimum, and maximum. Mean plasma concentration versus scheduled time profiles will be presented in figures on both linear and semilogarithmic scales. Individual plasma concentration versus actual time profiles will be presented similarly.

Pharmacokinetic parameters derived from plasma concentrations of total molybdenum and PUF molybdenum (if measured) will be presented in data listings and summarized separately using the following descriptive statistics: number of participants, arithmetic mean, GM, SD, arithmetic CV, GMCV, median, minimum, and maximum.

For PD (total and PUF (if measured) copper, NCC, LBC and NCC/NCC_{corrected}) and biomarker endpoints (ceruloplasmin, CpC), concentration versus time data will be listed and summarized with descriptive statistics and plotted. The same analyses will be conducted on the absolute and percent changes from Baseline of these concentration versus time data.

The following plasma PD parameters, as data permits, will be calculated for total copper, NCC, and LBC using noncompartmental methods with Phoenix® WinNonlin® Version 8.0 or higher or SAS Version 9.4 or higher, as applicable.
Maximum observed effect after dosing (CEₘₐₓ)
Time after dosing at which the maximum effect was observed (TEₘₐₓ)
Area under the effect versus time curve (AUEC) from the start of dose administration to the last observed quantifiable concentration (AUECₜ)

Pharmacodynamic parameters derived from plasma concentrations of total copper, NCC, and LBC will be presented and summarized by analyte and day similar to PK parameters.

Interim efficacy data will also be used in an efficacy extrapolation to develop evidence for BC-TTM efficacy on copper control for pediatric participants (ages 3 to <18 years) using adult/adolescent participant data from this and other phase 3 studies.

Population PK Simulations and Dose Selection in Pediatric Participants with WD

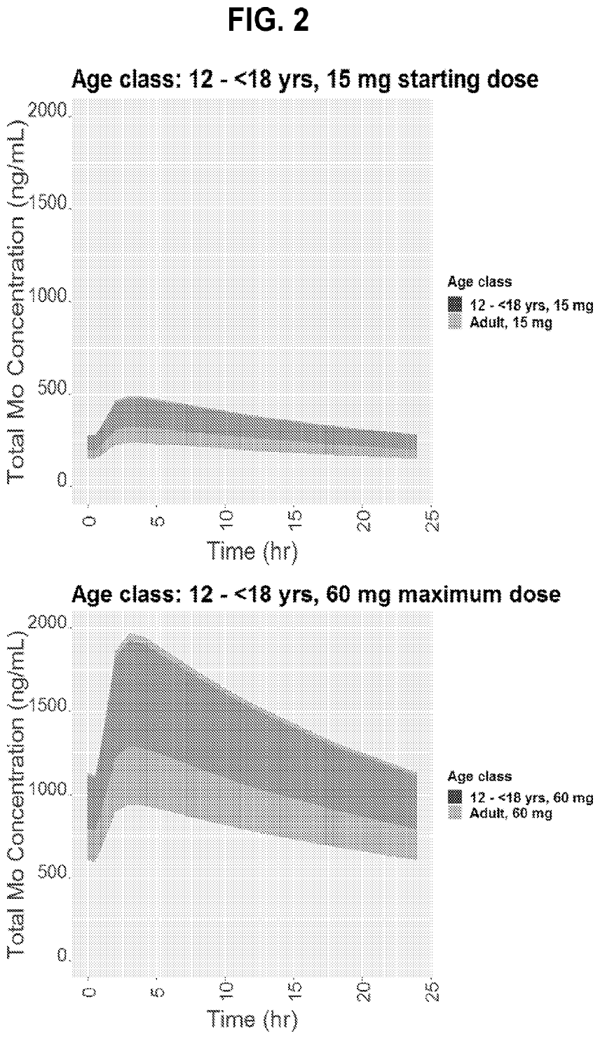

Participants aged 12 to <18 years: The simulation showed that projected PK exposure range for participants aged 12 to <18 years (FIG. 2) are expected to be overlapping with the exposure range for adults at the proposed dose of 15 mg in this and other Phase 3 studies.

The 15 mg EC tablet formulation is the current formulation being used for administration of the dose and subsequent individualized dose modifications in the ongoing Phase 3 Study-301, which includes participants aged 12 years and older, as per Protocol Amendment 1. The 15 mg/day daily dose has been shown to have favorable safety profiles and to be well tolerated throughout the Phase 1 to Phase 3 clinical studies in both healthy adults and adult participants with WD.

Of note, the current ongoing Phase 3 Study 301 has enrolled 17 participants aged 12 to <18 years, including a participant weighing 39.5 kg who was dosed at the starting dose of 15 mg BC-TTM once daily, and has shown an acceptable safety profile. To date, the TEAEs observed in the adolescent participants are similar to those reported in the overall study.

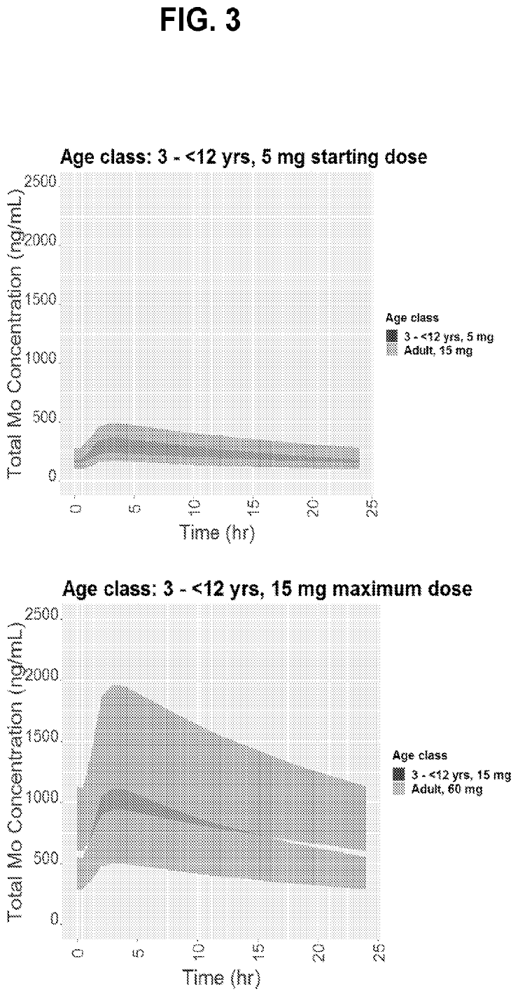

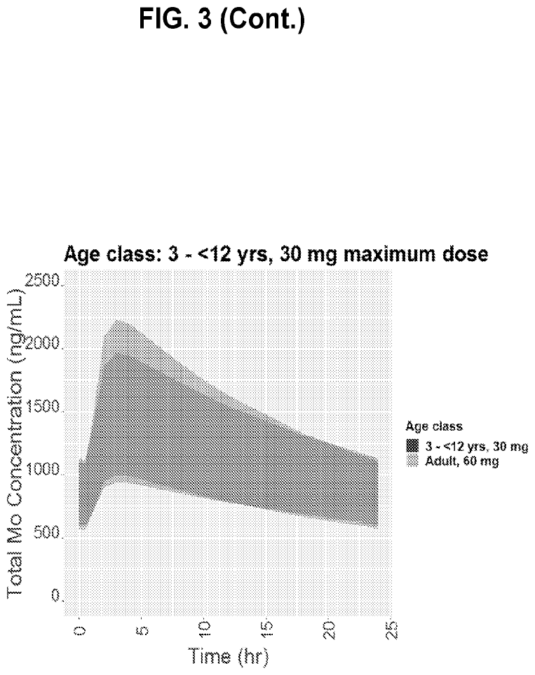

Participants aged 3 to <12 years: The projected PK exposures at the starting dose of 5 mg every other day are lower than those in adults administering a starting dose of 15 mg, a more conservative dosing approach in the youngest age group of 3 to <12 years. The projected exposures at the original maximum dose (i.e., 30 mg once daily) allowed for participants aged 3 to <12 years in Study 302 are similar to those administering the highest dose (i.e., 60 mg once daily) allowed in the ongoing Phase 3 Study 301 in adults and adolescents (FIG. 3).

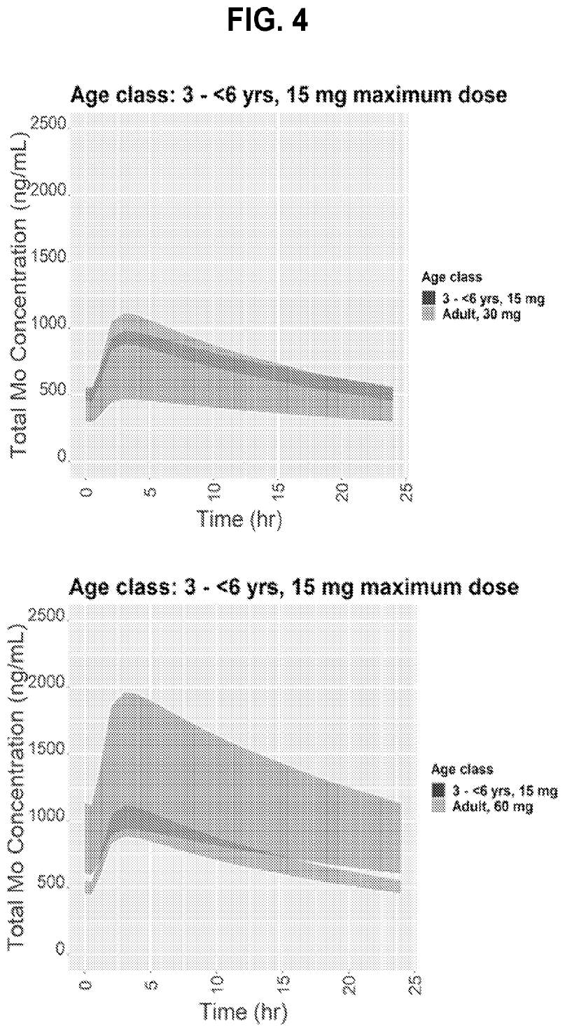

Looking specifically at participants aged 3 to <6 years, the modeling results showed that a 5 mg dose has comparable exposure as 15 mg administered to adult participants, and that a 15 mg dose has a higher exposure than 30 mg but a lower exposure than 60 mg administered to adult participants (FIG. 4). Hence, the predicted dose range for participants aged 3 to <6 years is 5 mg to 15 mg, which is anticipated to provide comparable safety and efficacy outcomes to those in adolescent and adult participants over 12 years of age with a dose range of 15 mg to 60 mg allowed in the ongoing Phase 3 Study −301.

A more conservative starting dose at 2.5 mg will be used for the present study 302 for participants aged 3 to <12 years.

It is understood that the examples and embodiments described herein are for illustrative purposes only and that various modifications or changes in light thereof are suggested to persons skilled in the art and are to be incorporated within the spirit and purview of this application and scope of the appended claims. All publications, patents, and patent applications cited herein are hereby incorporated herein by reference in their entirety for all purposes.

What is claimed is:

1: A method for treating a copper metabolism-associated disease or disorder in a subject, wherein the subject is from about 3 years old to less than about 18 years old, the method comprising administering to the subject a therapeutically effective amount of bis-choline tetrathiomolybdate.

2: The method of claim 1, wherein the copper metabolism-associated disease or disorder is Wilson Disease (WD).

3: The method of either claim 1 or claim 2, wherein the subject is from about 3 years old to less than about 12 years old.

4: The method of claim 3, wherein the therapeutically effective amount of bis-choline tetrathiomolybdate is in the range of about 2.5 mg to about 15 mg per day.

5: The method of claim 3, wherein the therapeutically effective amount of bis-choline tetrathiomolybdate is about 2.5 mg per day.

6: The method of claim 3, wherein the therapeutically effective amount of bis-choline tetrathiomolybdate is about 5 mg per day.

7: The method of claim 3, wherein the therapeutically effective amount of bis-choline tetrathiomolybdate is in the range of about 5 mg to about 15 mg per day.

8: The method of claim 5, wherein the therapeutically effective amount of bis-choline tetrathiomolybdate of about 2.5 mg per day is maintained for at least 4 weeks.

9: The method of claim 8, wherein, after at least 4 weeks, the therapeutically effective amount of bis-choline tetrathiomolybdate of about 2.5 mg per day is increased to a second therapeutically effective amount of bis-choline tetrathiomolybdate.

10: The method of claim 9, wherein the increase to the second therapeutically effective amount of bis-choline tetrathiomolybdate is in one or more increments of 2.5 mg per day, each increment at least 4 weeks apart.

11: The method of either claim 1 or claim 2, wherein the subject is from about 12 years old to less than about 18 years old.

12: The method of claim 11, wherein the therapeutically effective amount of bis-choline tetrathiomolybdate is in the range of about 15 mg every other day to about 15 mg per day.

13: The method of claim 11, wherein the therapeutically effective amount of bis-choline tetrathiomolybdate is about 15 mg every other day.

14: The method of claim 11, wherein the therapeutically effective amount of bis-choline tetrathiomolybdate is about 15 mg per day.

15: The method of claim 13, wherein the therapeutically effective amount of bis-choline tetrathiomolybdate of about 15 mg every other day is maintained for at least 4 weeks, for at least 8 weeks, for at least 12 weeks, for at least 24 weeks, for at least 48 weeks, for at least 72 weeks, or for at least 96 weeks, or for about 2 years, or for about 3 years, or for about 4 years, or for about 5 years, or for about 6 years, or for about 7 years, or for about 8 years, or for about 9 years, or for about 10 years or longer.

16: The method of claim 14, wherein the therapeutically effective amount of bis-choline tetrathiomolybdate of about 15 mg per day is maintained for at least 4 weeks, for at least 8 weeks, for at least 12 weeks, for at least 24 weeks, for at least 48 weeks, for at least 72 weeks, or for at least 96 weeks, or for about 2 years, or for about 3 years, or for about 4 years, or for about 5 years, or for about 6 years, or for about 7 years, or for about 8 years, or for about 9 years, or for about 10 years or longer.

17: The method of any one of claims 1 to 16, wherein the subject previously received no treatment for the copper metabolism-associated disease or disorder, such as for Wilson Disease (i.e., a treatment-naïve subject).

18: The method of any one of claims 1 to 16, wherein the subject previously received a standard of care treatment for the copper metabolism-associated disease or disorder, such as for Wilson Disease.

19: The method of claim 18, wherein the subject previously received standard of care treatment for less than 28 days.

20: The method of claim 18, wherein the subject previously received standard of care treatment for at least 28 days, for at least 5 weeks, for at least 6 weeks, for at least 12 weeks, for at least 24 weeks, or for at least 48 weeks.

21: The method of any one of claims 1 to 16, wherein the subject previously received no treatment or the subject previously received a standard of care treatment for less than 28 days for the copper metabolism-associated disease or disorder, such as for Wilson Disease.

22: The method of any one of claims 18 to 21, wherein the standard of care treatment comprises trientine, D-penicillamine, and/or zinc.

23: The method of any one of claims 18 to 21, wherein the standard of care treatment comprises trientine and/or D-penicillamine.

24: The method of any one of claims 18 to 23, wherein the subject received a last dose of the standard of care treatment at least 2 weeks prior to administering bis-choline tetrathiomolybdate.

25: The method of any one of claims 1 to 24, further comprising determining a concentration of one or more of total copper, ceruloplasmin, ceruloplasmin-bound copper (CpC), non-ceruloplasmin-bound copper (such as calculated, cNCC, or directly measured, dNCC), and labile-bound copper (LBC) in the subject's plasma.

26: The method of any one of claims 1 to 25, further comprising determining a concentration of NCC_{corrected}; or determining a daily mean area under the effect-time curve (AUEC) of directly measured non-ceruloplasmin-bound copper (dNCC) (e.g., from baseline to 48 weeks).

27: The method of claim 26, further comprising adjusting the therapeutically effective amount of bis-choline tetrathiomolybdate if the subject's NCC_{corrected} is outside a reference range for NCC_{corrected}.

28: The method of claim 27, wherein the reference range for NCC_{corrected} is 0.8 to 2.3 μM.

29: The method of any one of claims 1 to 28, further comprising determining a concentration of total molybdenum and/or plasma ultrafiltrate (PUF) molybdenum in the subject's plasma.

30: The method of any one of claims 25 to 29, wherein determining is performed at baseline, at or after 6 weeks of administration, at or after 24 weeks of administration, and/or at or after 48 weeks of administration.

31: The method of any one of claims 25 to 29, wherein determining is performed at baseline, and to 6 weeks of administration, or to 24 weeks of administration, or to 48 weeks of administration, or to at least 48 weeks or more of administration.

32: The method of any one of claims 1 to 31, further comprising evaluating the patients for improvements in disability and neurologic symptoms as measured according to Unified Wilson Disease Rating Scale (UWDRS), part II, and/or part Ill.

33: The method of any one of claims 1 to 32, further comprising evaluating the patients for improvements in disability status, psychiatric symptoms, clinical symptoms, treatment satisfaction, or a combination thereof.

34: A composition comprising bis-choline tetrathiomolybdate for use in the treatment of a copper metabolism-associated disease or disorder in a subject, wherein the subject is from about 3 years old to less than about 18 years old.

35: The composition of claim 34, wherein the copper metabolism-associated disease or disorder is Wilson Disease (WD).

36: The composition of either claim 34 or claim 35, wherein the subject is from about 3 years old to less than about 12 years old.

37: The composition of claim 36, wherein about 2.5 mg to about 15 mg per day of bis-choline tetrathiomolybdate is administered to the subject.

38: The composition of claim 36, wherein about 2.5 mg per day of bis-choline tetrathiomolybdate is administered to the subject.

39: The composition of claim 36, wherein about 5 mg per day of bis-choline tetrathiomolybdate is administered to the subject.

40: The composition of claim 36, wherein about 5 mg to about 15 mg per day of bis-choline tetrathiomolybdate is administered to the subject.

41: The composition of claim 38, wherein about 2.5 mg per day of bis-choline tetrathiomolybdate is administered to the subject for at least 4 weeks.

42: The composition of claim 41, wherein the amount of bis-choline tetrathiomolybdate that is administered to the subject is increased after at least 4 weeks.

43: The composition of claim 42, wherein the amount of bis-choline tetrathiomolybdate is increased by one or more increments of 2.5 mg per day.

44: The composition of either claim 34 or claim 35, wherein the subject is from about 12 years old to less than about 18 years old.

45: The composition of claim 44, wherein about 15 mg every other day to about 15 mg per day of bis-choline tetrathiomolybdate is administered to the subject.

46: The composition of claim 44, wherein about 15 mg of bis-choline tetrathiomolybdate is administered to the subject every other day.

47: The composition of claim 44, wherein about 15 mg per day of bis-choline tetrathiomolybdate is administered to the subject.

48: The composition of claim 46, wherein about 15 mg of bis-choline tetrathiomolybdate is administered to the subject every other day for at least 4 weeks, for at least 8 weeks, for at least 12 weeks, for at least 24 weeks, for at least 48 weeks, for at least 72 weeks, or for at least 96 weeks, or for about 2 years, or for about 3 years, or for about 4 years, or for about 5 years, or for about 6 years, or for about 7 years, or for about 8 years, or for about 9 years, or for about 10 years or longer.

49: The composition of claim 47, wherein about 15 mg per day of bis-choline tetrathiomolybdate is administered to the subject for at least 4 weeks, for at least 8 weeks, for at least 12 weeks, for at least 24 weeks, for at least 48 weeks, for at least 72 weeks, or for at least 96 weeks, or for about 2 years, or for about 3 years, or for about 4 years, or for about 5 years, or for about 6 years, or for about 7 years, or for about 8 years, or for about 9 years, or for about 10 years or longer.

50: The composition of any one of claims 34 to 49, wherein the subject previously received no treatment for the copper metabolism-associated disease or disorder, such as for Wilson Disease, (i.e., a treatment-naïve subject).

51: The composition of any one of claims 34 to 49, wherein the subject previously received a standard of care treatment for the copper metabolism-associated disease or disorder, such as for Wilson Disease.

52: The composition of claim 51, wherein the subject previously received standard of care treatment for less than 28 days.

53: The composition of claim 51, wherein the subject previously received standard of care treatment for at least 28 days, for at least 5 weeks, for at least 6 weeks, for at least 12 weeks, for at least 24 weeks, or for at least 48 weeks.

54: The composition of any one of claims 34 to 49, wherein the subject previously received no treatment or the subject previously received a standard of care treatment for less than 28 days for the copper metabolism-associated disease or disorder, such as for Wilson Disease.

55: The composition of any one of claims 51 to 54, wherein the standard of care treatment comprises trientine, D-penicillamine, and/or zinc.

56: The composition of any one of claims 51 to 54, wherein the standard of care treatment comprises trientine and/or D-penicillamine.

57: The composition of any one of claims 51 to 56, wherein the subject received a last dose of the standard of care treatment at least 2 weeks prior to receiving bis-choline tetrathiomolybdate.

58: The composition of any one of claims 34 to 57, wherein the concentration of one or more of total copper, ceruloplasmin, ceruloplasmin-bound copper (CpC), non-ceruloplasmin-bound copper (such as calculated, cNCC, or directly measured, dNCC), and labile-bound copper (LBC) in the subject's plasma is determined.

59: The composition of any one of claims 34 to 58, wherein the concentration of NCC_{corrected} or the daily mean area under the effect-time curve (AUEC) of directly measured non-ceruloplasmin-bound copper (dNCC) (e.g., from baseline to 48 weeks) is determined.

60: The composition of claim 59, wherein the amount of bis-choline tetrathiomolybdate is adjusted if the subject's NCC_{corrected} is outside a reference range for NCC_{corrected}.

61: The composition of claim 60, wherein the reference range for NCC_{corrected} is 0.8 to 2.3 μM.

62: The composition of any one of claims 34 to 61, wherein the concentration of total molybdenum and/or plasma ultrafiltrate (PUF) molybdenum in the subject's plasma is determined.

63: The composition of any one of claims 58 to 62, wherein the concentration of one or more of total copper, ceruloplasmin, ceruloplasmin-bound copper (CpC), non-ceruloplasmin-bound copper, and labile-bound copper (LBC) in the subject's plasma is determined at baseline, at or after 6 weeks of administration of bis-choline tetrathiomolybdate, at or after 24 weeks of administration of bis-choline tetrathiomolybdate, and/or at or after 48 weeks of administration of bis-choline tetrathiomolybdate.

64: The composition of any one of claims 58 to 62, wherein the concentration of one or more of total copper, ceruloplasmin, ceruloplasmin-bound copper (CpC), non-ceruloplasmin-bound copper, and labile-bound copper (LBC) in the subject's plasma is determined at baseline, and to 6 weeks of administration of bis-choline tetrathiomolybdate, or to 24 weeks of administration of bis-choline tetrathiomolybdate, or to 48 weeks of administration, or to at least 48 weeks or more of administration of bis-choline tetrathiomolybdate.

65: The composition of any one of claims 34 to 64, wherein the subject is evaluated for improvements in disability and neurologic symptoms as measured according to Unified Wilson Disease Rating Scale (UWDRS), part II, and/or part Ill.

66: The composition of any one of claims 34 to 65, wherein the subject is evaluated for improvements in disability status, psychiatric symptoms, clinical symptoms, treatment satisfaction, or a combination thereof.

67: Use of a composition comprising bis-choline tetrathiomolybdate for the manufacture of a medicament for treating a copper metabolism-associated disease or disorder in a subject, wherein the subject is from about 3 years old to less than about 18 years old.

68: The use of claim 67, wherein the copper metabolism-associated disease or disorder is Wilson Disease (WD).

69: The use of either claim 67 or claim 68, wherein the subject is from about 3 years old to less than about 12 years old.

70: The use of claim 69, wherein about 2.5 mg to about 15 mg per day of bis-choline tetrathiomolybdate is administered to the subject.

71: The use of claim 69, wherein about 2.5 mg per day of bis-choline tetrathiomolybdate is administered to the subject.

72: The use of claim 69, wherein about 5 mg per day of bis-choline tetrathiomolybdate is administered to the subject.

73: The use of claim 69, wherein about 5 mg to about 15 mg per day of bis-choline tetrathiomolybdate is administered to the subject.

74: The use of claim 71, wherein about 2.5 mg per day of bis-choline tetrathiomolybdate is administered to the subject for at least 4 weeks.

75: The use of claim 74, wherein the amount of bis-choline tetrathiomolybdate that is administered to the subject is increased after at least 4 weeks.

76: The use of claim 75, wherein the amount of bis-choline tetrathiomolybdate is increased by one or more increments of 2.5 mg per day.

77: The use of either claim 67 or claim 68, wherein the subject is from about 12 years old to less than about 18 years old.

78: The use of claim 77, wherein about 15 mg every other day to about 15 mg per day of bis-choline tetrathiomolybdate is administered to the subject.

79: The use of claim 77, wherein about 15 mg of bis-choline tetrathiomolybdate is administered to the subject every other day.

80: The use of claim 77, wherein about 15 mg per day of bis-choline tetrathiomolybdate is administered to the subject.

81: The use of claim 79, wherein about 15 mg of bis-choline tetrathiomolybdate is administered to the subject every other day for at least 4 weeks, for at least 8 weeks, for at least 12 weeks, for at least 24 weeks, for at least 48 weeks, for at least 72 weeks, or for at least 96 weeks, or for about 2 years, or for about 3 years, or for about 4 years, or for about 5 years, or for about 6 years, or for about 7 years, or for about 8 years, or for about 9 years, or for about 10 years or longer.

82: The use of claim 80, wherein about 15 mg per day of bis-choline tetrathiomolybdate is administered to the subject for at least 4 weeks, for at least 8 weeks, for at least 12 weeks, for at least 24 weeks, for at least 48 weeks, for at least 72 weeks, or for at least 96 weeks, or for about 2 years, or for about 3 years, or for about 4 years, or for about 5 years, or for about 6 years, or for about 7 years, or for about 8 years, or for about 9 years, or for about 10 years or longer.

83: The use of any one of claims 67 to 82, wherein the subject previously received no treatment for the copper metabolism-associated disease or disorder, such as for Wilson Disease, (i.e., a treatment-naïve subject).

84: The use of any one of claims 67 to 82, wherein the subject previously received a standard of care treatment for the copper metabolism-associated disease or disorder, such as for Wilson Disease.

85: The use of claim 84, wherein the subject previously received standard of care treatment for less than 28 days.

86: The use of claim 84, wherein the subject previously received standard of care treatment for at least 28 days, for at least 5 weeks, for at least 6 weeks, for at least 12 weeks, for at least 24 weeks, or for at least 48 weeks.

87: The use of any one of claims 67 to 82, wherein the subject previously received no treatment or the subject previously received a standard of care treatment for less than 28 days for the copper metabolism-associated disease or disorder, such as for Wilson Disease.

88: The use of any one of claims 84 to 87, wherein the standard of care treatment comprises trientine, D-penicillamine, and/or zinc.

89: The use of any one of claims 84 to 87, wherein the standard of care treatment comprises trientine and/or D-penicillamine.

90: The use of any one of claims 84 to 89, wherein the subject received a last dose of the standard of care treatment at least 2 weeks prior to receiving bis-choline tetrathiomolybdate.

91: The use of any one of claims 67 to 90, wherein the concentration of one or more of total copper, ceruloplasmin, ceruloplasmin-bound copper (CpC), non-ceruloplasmin-bound copper (such as calculated, cNCC, or directly measured, dNCC), and labile-bound copper (LBC) in the subject's plasma is determined.

92: The use of any one of claims 67 to 91, wherein the concentration of NCC_{corrected} or the daily mean area under the effect-time curve (AUEC) of directly measured non-ceruloplasmin-bound copper (dNCC) (e.g., from baseline to 48 weeks) is determined.

93: The use of claim 92, wherein the amount of bis-choline tetrathiomolybdate is adjusted if the subject's NCC_{corrected} is outside a reference range for NCC_{corrected}.

94: The use of claim 93, wherein the reference range for NCC_{corrected} is 0.8 to 2.3 μM.

95: The use of any one of claims 67 to 94, wherein the concentration of total molybdenum and/or plasma ultrafiltrate (PUF) molybdenum in the subject's plasma is determined.

96: The use of any one of claims 91 to 95, wherein the concentration of one or more of total copper, ceruloplasmin, ceruloplasmin-bound copper (CpC), non-ceruloplasmin-bound copper, and labile-bound copper (LBC) in the subject's plasma is determined at baseline, at or after 6 weeks of administration of bis-choline tetrathiomolybdate, at or after 24 weeks of administration of bis-choline tetrathiomolybdate, and/or at or after 48 weeks of administration of bis-choline tetrathiomolybdate.

97: The use of any one of claims 91 to 95, wherein the concentration of one or more of total copper, ceruloplasmin, ceruloplasmin-bound copper (CpC), non-ceruloplasmin-bound copper, and labile-bound copper (LBC) in the subject's plasma is determined at baseline, and to 6 weeks of administration of bis-choline tetrathiomolybdate, or to 24 weeks of administration of bis-choline tetrathiomolybdate, or to 48 weeks of administration, or to at least 48 weeks or more of administration of bis-choline tetrathiomolybdate.

98: The use of any one of claims 67 to 97, wherein the subject is evaluated for improvements in disability and neurologic symptoms as measured according to Unified Wilson Disease Rating Scale (UWDRS), part II, and/or part Ill.

99: The use of any one of claims 67 to 98, wherein the subject is evaluated for improvements in disability status, psychiatric symptoms, clinical symptoms, treatment satisfaction, or a combination thereof.
